(19) United States
(12) Patent Application Publication
(10) Pub. No.: US 2018/0055408 A1
(43) Pub. Date: Mar. 1, 2018

(54) QUANTITATIVE DIFFERENTIATION OF INFLAMMATION FROM SOLID TUMORS, HEART AND NERVE INJURY

(71) Applicant: Washington University, St. Louis, MO (US)

(72) Inventors: Sheng-Kwei Song, St. Louis, MO (US); Zezhong Ye, St. Louis, MO (US); William Spees, St. Louis, MO (US); Tsen-Hsuan Lin, St. Louis, MO (US)

(21) Appl. No.: 15/691,119

(22) Filed: Aug. 30, 2017

Related U.S. Application Data
(60) Provisional application No. 62/381,172, filed on Aug. 30, 2016.

(51) Int. Cl. A61B 5/055; G01R 33/563; G01R 33/56; A61B 5/00; A61B 10/02; A61B 5/20; A61B 10/00; G06T 7/00

(52) U.S. Cl. CPC A61B 5/055; A61B 2010/045; G01R 33/5608; A61B 5/7267; A61B 5/4381; A61B 10/0241; A61B 5/0042; A61B 5/0044; A61B 5/201; A61B 5/202; A61B 5/4041; A61B 5/425; A61B 5/4312; A61B 5/4331; A61B 10/0041; G06T 7/0012; A61B 2576/023; A61B 2576/026; G01R 33/56341

(57) ABSTRACT

D-Histo, a non-invasive diagnostic method, renovated from diffusion basis spectrum imaging (DBSI) is provided for quantitatively detecting and distinguishing inflammation from solid tumors, heart and nerve injury. For example, the D-Histo methods disclosed herein provide an accurate diagnosis of prostate cancer, distinguishing it from prostatitis and BPH that missed by currently available methods of diagnosing prostate cancer (multiparameter MRI, needle biopsy). The disclosed D-Histo method also provides metrics to reflect reversible vs. irreversible damages in heart and central/peripheral nerves. For central and peripheral nerves, D-Histo also provides metrics to assess nerve functionality. The at least one D-Histo biomarker obtained using diffusion weighted MRI has excellent test-retest stability, high sensitivity to disease progression and close correlation with currently available techniques.

### PRIORITY CLAIM

This application claims priority to U.S. Provisional Application No. 62/381,172, filed on Aug. 30, 2016. The entirety of that application is hereby incorporated by reference.

### STATEMENT REGARDING FEDERALLY SPONSORED RESEARCH & DEVELOPMENT

This invention was made with government support under grant NS059560 awarded by the National Institutes of Health. The U.S. government has certain rights in the invention.

### BACKGROUND

Aspects of the disclosure relate generally to quantitative differentiation of inflammation and solid tumor as a diagnostic approach to the characterization of prostate cancer.

### BRIEF DESCRIPTION

Prostate cancer (PCa) is the most prevalent malignancy afflicting American men and accounts for over 26,000 deaths annually. Current imaging modalities do not distinguish PCa from prostatitis (prostate gland inflammation) or benign prostatic hyperplasia (BPH). As a result of this blind spot, perplexing diagnoses can arise when inflammatory biopsy specimens free of PCa are scored as PCa positive according to Prostate Imaging-Reporting and Data System (PI-RADS) version 2 guidelines. The contribution of inflammatory cells to the prostate apparent diffusion coefficient (ADC) measurement has never been determined. Inflammatory and prostate cancer cells coexist leading to false-positive identification of PCa by the recently established PI-RADS guideline and the use of multiparametric MRI (mpMRI) for PCa diagnosis.

Methods and systems disclosed herein utilize modifications of diffusion basis spectrum imaging (DBSI) as a tool to image the prostate gland and identify structural and size-related cell differences. As a result, PCa, prostatitis, and BPH can be detected, distinguished from one another, and individually quantified without the need to inject exogenous contrast agents. As will be explained, other types of microstructures may be detected and distinguished from each other. False positive identification of cancerous cells may therefore be reduced.

### SUMMARY

One example disclosed is a method for diagnosing at least one prostate disorder in a patient utilizing diffusion-weighted MRI (D-Histo). Diffusion-weighted MRI data is captured for a plurality of voxels within at least a portion of a prostate of the patient. At least one D-Histo biomarker is identified in the captured diffusion-weighted MRI data. The at least one prostate disorder is detected in each voxel of the plurality based on the at least one D-Histo biomarker. The at least one detected prostate disorder is quantified based on the at least one D-Histo biomarker. The at least one prostate disorder is selected from at least one of: prostate cancer (PCa), prostatitis, and benign stromal hyperplasia (BPH).

Another example is a method for diagnosing at least one cardiac disorder in a patient utilizing diffusion-weighted MRI (D-Histo). Diffusion-weighted MRI data is captured for a plurality of voxels within at least a portion of a heart of the patient. At least one D-Histo biomarker is identified in the captured diffusion-weighted MRI data. The at least one cardiac disorder is detected in each voxel based on the at least one D-Histo biomarker. The at least one detected cardiac disorder is quantified based on the at least one D-Histo biomarker. The at least one cardiac disorder is selected from at least one of myocarditis and myocardial infarction.

Another example is a method for diagnosing at least one disorder of a cervix in a patient utilizing diffusion-weighted MRI (D-Histo). Diffusion-weighted MRI data is captured for a plurality of voxels within at least a portion of the cervix of the patient. At least one D-Histo biomarker is identified in the captured diffusion-weighted MRI data. The at least one disorder of the cervix is detected in each voxel based on the at least one D-Histo biomarker. The at least one detected disorder of the cervix is quantified based on the at least one D-Histo biomarker. At least one disorder of the cervix is selected from at least one of cervical cancer and inflammation of the cervix.

Another example is a method for diagnosing at least one brain disorder in a patient utilizing diffusion-weighted MRI (D-Histo). Diffusion-weighted MRI data is captured for a plurality of voxels within at least a portion of a brain of the patient. At least one D-Histo biomarker is identified in the captured diffusion-weighted MRI data. The at least one brain disorder is detected in each voxel based on the at least one D-Histo biomarker. The at least one detected brain disorder is quantified based on the at least one D-Histo biomarker. The at least one brain disorder is selected from at least one of: brain inflammation, brain cancer, and brain cancer with tumor necrosis.

Another example is a method for diagnosing at least one central or peripheral nerve disorder in a patient utilizing diffusion-weighted MRI (D-Histo). Diffusion-weighted MRI data is captured for a plurality of voxels within at least a portion of a central or peripheral nerve of the patient. At least one D-Histo biomarker is identified in the captured diffusion-weighted MRI data. The at least one central or peripheral nerve disorder is detected in each voxel of the plurality based on the at least one D-Histo biomarker. The at least one detected central or peripheral nerve disorder is quantified based on the at least one D-Histo biomarker. The at least one central or peripheral nerve disorder is selected from at least one of: inflammation, edema, demyelination and axonal injury/loss.

Another example is a method of classifying microstructures in a tissue volume. An MRI of the tissue volume is taken by an MRI scanner. Diffusion tensor components of water molecules are determined within a voxel derived from the MRI image via a processor coupled to the MRI scanner. The apparent diffusion coefficients of the water molecules are determined for the diffusion tensor components falling in a predetermined range associated with a microstructure via the processor. The microstructure is identified in the voxel derived from the MRI image based on classified diffusion tensor components where the apparent diffusion coefficients fall in the predetermined range via the processor.

Another example is a method of differentiating cancerous and non-cancerous microstructures in a tissue volume. An MRI image is taken of the tissue volume by an MRI scanner. Diffusion tensor components from the MRI image are determined via a processor coupled to the MRI scanner. Diffusion tensor components of water molecules are determined within a voxel derived from the MRI image via the processor. The apparent diffusion coefficients of the water molecules are determined for the diffusion tensor components falling in a predetermined range associated with a cancerous microstructure via the processor. The cancerous microstructure in the voxel derived from the MRI image is identified based on classified diffusion tensor components where the apparent diffusion coefficients fall in the predetermined range associated with the cancerous microstructure via the processor. The apparent diffusion coefficients of the water molecules are applied for the diffusion tensor components falling in a predetermined range associated with a non-cancerous microstructure via the processor. The non-cancerous microstructure in the voxel derived from the MRI image are identified based on classifying diffusion tensor components where the apparent diffusion coefficients fall in the predetermined range associated with the non-cancerous microstructure via the processor.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an illustration of diffusion magnetic resonance (MR) signal response when diffusion tensor imaging (DTI) is applied to a single white matter tract of coherent axonal fibers.

FIG. 2 is an illustration of exemplary DTI results corresponding to scenarios in which different tissue components are included within a scanned volume.

FIG. 3A is a flowchart of an exemplary noninvasive process to quantify complex CNS white matter pathology.

FIGS. 3B and 3C are a flowchart of an exemplary method for determining diffusivities of fibers and isotropic components within a tissue.

FIGS. 4A-4B are illustrations of the design of an exemplary 99-direction diffusion-weighting scheme.

FIG. 5 is an illustration of an exemplary diffusion basis set for DBSI.

FIG. 6 is an illustration of an exemplary optimization process of DBSI basis set.

FIGS. 7A-7B are illustrations of determining the number of fibers and primary directions of candidate fibers using DBSI.

FIG. 8 is an illustration of an exemplar optimization process for determining the directional diffusivity of each candidate fiber, isotropic components and corresponding volume ratios using DBSI.

FIG. 9 is an illustration of diffusion basis spectrum imaging (DBSI) results reflecting demyelination as increased radial diffusivity in the presence of axonal injury, and inflammation in contrast to the failure of DTI to detect the pathology.

FIG. 10 is a graph of myelin basic protein in a MBP-positive area derived from DBSI of FIG. 9.

FIG. 11 is a graph of the radial diffusivity derived using DBSI of FIG. 9.

FIG. 12 is an illustration of DBSI results detecting axonal injury in the presence of demyelination and inflammation.

FIG. 13 is a graph of the axial diffusivity of the DBSI of FIG. 12.

FIG. 14 is a graph of the SMI-31 stain of the DBSI of FIG. 12.

FIG. 15 is a graph of axonal fiber tract density was also derived using DBSI expressing as volume ratio of the DBSI of FIG. 12.

FIG. 16 is an illustration of DBSI results quantifying inflammation in the presence of axonal injury and demyelination.

FIG. 17 is a graph of the percentage of inflammatory cell infiltration thought to be in the cells of the illustration of FIG. 16.

FIG. 18 is an exemplary phantom of mouse trigeminal nerves embedded in gel with known in vivo DTI character.

FIG. 19 is a comparison of diffusion spectrum imaging (DSI) and DBSI from a human subject.

FIG. 20 is a diffusion tensor imaging (DTI) for mouse trigeminal nerve embedded in gel.

FIG. 21 is a DBSI for mouse trigeminal nerve embedded in gel.

FIG. 22 is a graph of the nucleus and axon counts by IHC of FIG. 29.

FIG. 23 is a graph of the DBSI derived cell percentage and fiber percentage of FIG. 22.

FIG. 24 is an illustration of a typical DBSI-derived spectrum of isotropic diffusivity from a fixed mouse trigeminal nerve juxtaposed with gel.

FIG. 25 is a comparison of DBSI-derived gel fractions to those measured by T2W MRI signal intensity.

FIG. 26 is a graph of λ_{∥} derived from trigeminal nerves with and without gel.

FIG. 27 is a graph of λ_{⊥} derived from trigeminal nerves with and without gel.

FIG. 28 is an illustration of six fixed trigeminal nerves grouped into three pairs of crossing fibers at 32°, 58°, and 91° juxtaposed with 2% agarose gel.

FIG. 29 is an illustration of a three-fiber crossing phantom forming a triangle.

FIG. 30 is an illustration of in vivo DBSI derived λ_{∥}, λ_{⊥}, and cell fraction maps of mice from each time point are displayed with the corresponding axon (SMI-31), myelin (MBP), and nucleus (DAPI) staining.

FIG. 31 is a cross-sectional time course of in vivo DBSI derived λ_{∥} from B6-EAE mice at baseline (control), onset, peak, and chronic disease states.

FIG. 32 is a cross-sectional time course of in vivo DBSI derived λ_{⊥} from B6-EAE mice at baseline (control), onset, peak, and chronic disease states.

FIG. 33 is a cross-sectional time course of in vivo DBSI derived cell intensity percentage from B6-EAE mice at baseline (control), onset, peak, and chronic disease states.

FIG. 34 is a cross-sectional time course of in vivo DBSI derived water intensity percentage from B6-EAE mice at baseline (control), onset, peak, and chronic disease states.

FIG. 35 is an illustration of a targeted prostate cancer biopsy.

FIG. 36 shows different types of water molecular diffusion.

FIG. 37A is a 3-D reconstruction of a prostate in a transverse view showing the needle biopsy locations.

FIG. 37B is a 3-D reconstruction of a prostate in a sagittal view showing the needle biopsy locations.

FIGS. 38A-38C show mpMRI images obtained from a PCa patient.

FIG. 39A-39B are microscope images with a pathologist-marked region of PCa.

FIG. 40 is an illustration of diffusion MRI histology model components.

FIG. 41 is a series of MRI based images showing an example comparison between mpMRI and D-Histo results for PCa detection.

FIG. 42 is a series of microscopic images of a whole mount section H & E stain.

FIG. 43 shows diffusion fraction results for peripheral zone and central gland PCa.

FIG. 44 is a 3-D reconstruction of a prostate showing regions of inflammation and cancerous regions.

FIG. 45 is an illustration of components of a voxel volume within a prostate.

FIG. 46 shows graphs of PCa volume and percentage of total prostate volume results with respect to D-Histo PCa detection.

FIG. 47 shows images of a comparison of in vivo mpMRI and D-Histo measurements of a prostate with transition zone PCa.

FIG. 48 shows images of in vivo mpMRI and D-Histo measurements of a prostate with peripheral and transition zone PCa.

FIG. 49 is a 3D-rendered prostate overlaid on an anatomical image.

FIG. 50 shows images of a whole mount H & E slide with corresponding D-Histo maps.

FIG. 51 is an example comparison between mpMRI/DBSI and D-Histo for normal and PCa prostates.

FIG. 52 is another example comparison between mpMRI/DBSI and D-Histo for normal and PCa prostates.

FIG. 53 is yet another example comparison between mpMRI/DBSI and D-Histo for normal and PCa prostates.

FIG. 54 is an additional example comparison between mpMRI/DBSI and D-Histo for normal and PCa prostates.

FIG. 55 is a series of images illustrating existing imaging technologies for diagnosing brain tumors.

FIG. 56 is a microscope image of brain tissue containing various cell types including red blood cells, lymphocytes, invading tumor cells, necrotic tumor cells, low grade gliomas, and glioblastomas.

FIG. 57 shows images from a case of anaplastic ependymoma illustrating isotropic diffusion spectra characterizing white matter, brain tumor cells, and blood cells.

FIG. 58 is a series of images comparing DBSI and D-Histo analysis of the same brain tissue.

FIG. 59 is a series of images comparing D-Histo metric maps to existing H&E histological images and MRI images of the same brain tissue.

FIG. 60 is a series of images comparing a map of restricted and hindered isotropic diffusion fraction to existing Ki-67 stained histological images of same brain tissue.

FIG. 61 is a series of images from DBSI and D-Histo techniques in a rat heart of myocardial infarction.

FIG. 62 is a series of images from mpMRI and D-Histo techniques of cervical cancer.

FIG. 63 is a series of images from mpMRI and D-Histo techniques of cervical cancer.

FIG. 64 shows a series of MR images and histological staining for a pediatric primitive neuroectodermal tumor (PNET).

FIG. 65 is a series of distributions showing the process of creating a quantitative histology map.

FIG. 66 shows classification of tissue analyzed by SVM based on a training set from histology classification.

FIG. 67 shows DTI and D-Histo derived parametric maps of one representative EAE optic nerve of a mouse.

FIG. 68 shows diffusion-weighted images (DWI) acquired using the diffusion gradient applied perpendicular to the optic nerves of a mouse.

FIG. 69 shows a series of graphs quantitatively validating D-Histo derived pathological metrics.

FIG. 70 shows the image planning to obtain the cross-sectional view of one optic nerve.

FIG. 71 show images that demonstrate that D-Histo and DTI derived radial diffusivity of optic nerve decreased upon visual stimulation and D-Histo derived restricted fraction maps upon visual stimulation.

FIG. 72 is a schematic block diagram of an MRI imaging system in one aspect.

FIG. 73 is a schematic block diagram of an example server system.

FIG. 74 is a block diagram of an example computing device.

### DETAILED DESCRIPTION

For the context of the present disclosure, an in-depth discussion of diffusion MRI is first provided, followed by a detailed description of quantitative differentiation of inflammation and solid tumors.

Abbreviations: MRI, magnetic resonance imaging; DBSI, diffusion basis spectrum imaging; dMRI, diffusion MRI; DTI, diffusion tensor imaging; PCa, prostate cancer; mpMRI, multi-parametric MRI; BPH, benign prostatic hyperplasia; ADC, apparent diffusion coefficient; PI-RADS, prostate imaging—reporting and data system; mDBSI, modified DBSI; CG, central gland; PZ, peripheral zone; DRE, digital rectal exam; PSA, prostate specific antigen; D-Histo, diffusion [MRI] histology; H&E staining, haemotoxylin and eosin staining; DWI, diffusion-weighted imaging; DCE, Dynamic Contrast Enhanced Imaging; T1W, T1-weighted imaging; T2W, T2-weighted imaging.

### Diffusion MRI

The following discussion relates generally to magnetic resonance imaging (MRI) and, more particularly, to diffusion magnetic resonance data provided by an MRI scanner.

White matter injury is common in central nervous system (CNS) disorders and plays an important role in neurological dysfunctions in patients. Understanding the pathology of complex and heterogeneous central nervous system diseases such as multiple sclerosis (MS) has been greatly hampered by the dearth of histological specimens obtained serially during the disease. Clinicians are reluctant to perform invasive CNS biopsies on patients with white matter disorders, due to the potential injury to the patients.

The insight of CNS white matter neuropathology has been derived typically from occasional biopsies consisting of small tissue samples of unusual cases. These autopsies usually derive from patients with end-stage disease and often have long postmortem delay artifacts due to tissue degradation. It is therefore advantageous to have a noninvasive imaging tool to accurately quantify and better understand the chronic and non-fatal injury in CNS disease during the whole course of the individual patient.

Diffusion tensor imaging (DTI) is a commonly used MRI modality in CNS disease/injury diagnosis. However, the current use of DTI technique is not capable of resolving the complex underlying pathologies correctly, despite being considered better than other techniques.

A diffusion MRI technique is discussed herein to noninvasively study and quantify complicated CNS diseases in a noninvasive fashion without the limitation of invasive histological examinations.

Such embodiments facilitate improved results compared to diffusion tensor imaging (DTI). The directional diffusivities derived from DTI measurements describe water movement parallel to (λ_{∥}, axial diffusivity) and perpendicular to (λ_{⊥}, radial diffusivity) axonal tracts. It was previously proposed and validated that decreased λ_{∥} is associated with axonal injury and dysfunction, and increased λ_{⊥} is associated with myelin injury in mouse models of white matter injury.

The presence of inflammation, edema, or gliosis during CNS white matter injury may impact the DTI measurement. One significant effect of inflammation is the resulting isotropic component of diffusion, due to the increased extracellular water and the infiltrating immune cells. These components complicate the DTI measurements and distorts the estimated directional diffusivity and anisotropy preventing its accurate interpretation of underlying pathologies. In addition to inflammation, similar isotropic diffusion tensor component may result from the loss of CNS tissues in the chronic MS lesions, spinal cord injury (SCI), or traumatic brain injury (TBI). The currently used DTI protocol is not able to resolve this isotropic component or differentiate inflammation from tissue loss. Only an averaged diffusion tensor reflecting the overall effect can be obtained from existing DTI methods.

DTI fails to (1) correctly describe axonal fiber directions in crossing white matter tracts, or (2) accurately reflect the complex white matter pathologies such as vasogenic edema, inflammation, and tissue loss commonly coexisting with axonal and myelin damages. Even recently developed existing systems are not capable of resolving white matter pathologies in complex tissue scenarios.

A noninvasive process based on diffusion MRI technique is described herein to facilitate accurately quantifying the complex human CNS white matter pathology where the current DTI and its relevant improvements have failed. As an exemplary embodiment, diffusion basis spectrum imaging (DBSI) is implemented and provided herein to demonstrate the feasibility and detailed operation of the process. The quantity and primary direction of diffusion tensor components within a tissue volume resulting from white matter pathology is determined using diffusion MRI before constructing the multi-tensor model. After the identification of each diffusion tensor component corresponding to individual pathology, the diffusivity and volume ratio of each component can be derived accordingly.

In some embodiments, the quantity of candidate fibers and their associated primary directions are calculated first by DBSI based on a combination of diffusion basis set best describing the measured diffusion magnetic resonance data. An isotropic diffusion component is also considered to improve the computation accuracy. Based on all candidate fibers' primary directions, DBSI is used to compute the axial diffusivity, indicating water diffusion parallel to the fiber, and radial diffusivity, indicating water diffusion perpendicular to the fiber. A diffusivity spectrum of isotropic diffusion components, such as those resulting from inflammation or tissue loss, as well as associated volume ratios of all candidate fibers and isotropic components may be calculated.

An exemplary embodiment employs diffusion basis spectrum imaging (DBSI) to facilitate an accurate diagnosis of CNS white matter pathology. Each diffusion tensor's directional diffusivity as well as its primary orientation is derived using the less stringent diffusion tensor acquisition schemes retaining DTI's applicability in clinical settings. Preliminary data in mouse corpus callosum, spinal cord injury, and phantoms demonstrates that DBSI is capable of identifying different underlying pathologies accurately estimating the extent of cell infiltration, axonal fiber density in corpus callosum of cuprizone treatment, as well as estimating tissue loss in chronic mouse spinal cord injury. Diffusion phantoms have also been designed and fabricated for a quantitative evaluation of DBSI and existing DTI methods.

The exemplary embodiment of diffusion MRI described herein resolves the multi-tensor complication resulting from diverse pathologies in CNS white matter to quantitatively derive diffusion parameters of crossing fibers as well as reflecting the actual pathologies. This unique capability of the proposed process and the exemplary DBSI method has the potential to differentiate acute inflammation from chronic tissue loss in patients. Such capability can estimate the extent of acute inflammation guiding the use of anti-inflammatory treatment and chronic tissue damage guiding the effort in axonal/neuronal preservation. There are many potential clinical applications of the proposed process. For example, it can document the efficacy of stem cell treatment in axonal regeneration by clearly estimating the isotropic component of the implanted cells while reflecting the axonal regeneration by quantifying the anisotropic component changes after cell transplantation. It could also be used to estimate the degree of CNS tumor growth by accurately estimating the isotropic tensor component representing the tumor cells. Methods described further facilitate evaluating the effectiveness of a drug in treating one or more medical conditions. For example, DBSI could be applied in clinical drug trial treating CNS diseases, tumors, and injury by accurately reflecting the progression of clinical and preclinical pathologies.

One important characteristic of DTI is its ability to measure diffusion anisotropy of CNS tissues for a detailed description of the underlying tissue injury based on the changed diffusion character. However, such measurement is not always obtainable in diseased tissues due to the complicated cellular responses to the pathology or the presence of crossing fibers.

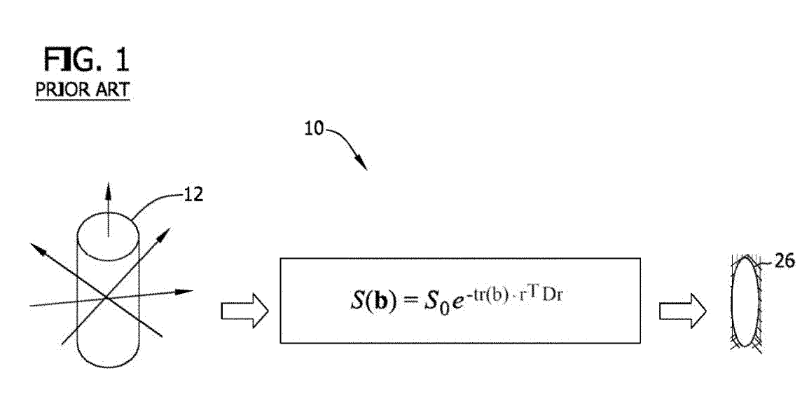

The fundamental operation of DTI 10 can be explained by examining an MRI signal 12 under the influence of diffusion weighting gradients. When applying DTI to measure the single white matter tract of coherent axonal fibers, the MRI signal response can be expressed as shown in FIG. 1.

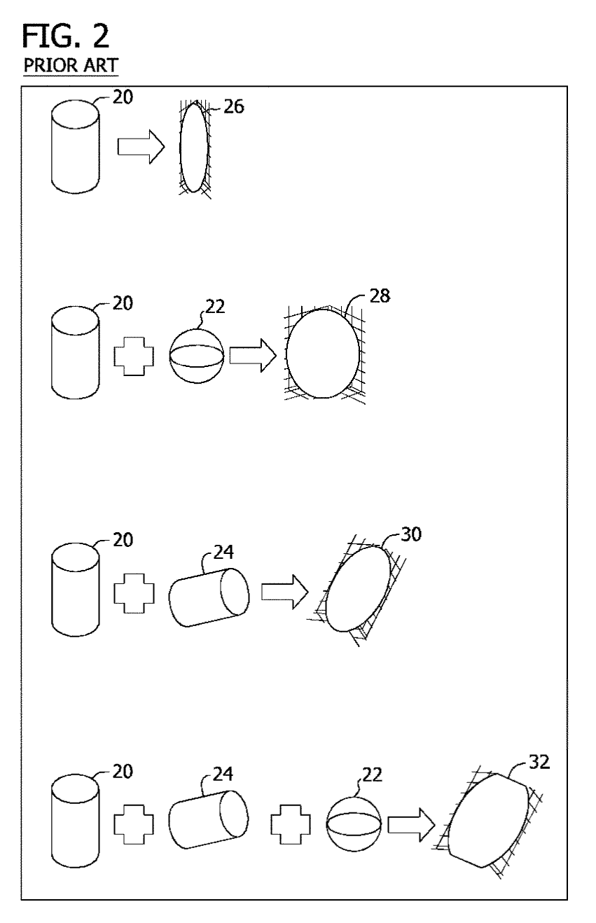

DTI assumes that there is only a pure coherent axonal fiber tract in the measured tissue and the signal response to diffusion weighting gradients is well described by the diffusion weighted (DW) signal profile. The insufficiency of DTI can be demonstrated by examining the diffusion ellipsoid responding to the different tissue components that typically seen in CNS tissues with and without pathology, as shown in FIG. 2.

FIG. 2 illustrates exemplary DTI results corresponding to scenarios with the different tissue components (objects), including (A) ideal coherent single fiber 20 (spinal cord white matter or optic nerves), (B) fiber 20 plus an isotropic component 22 (tissue loss, inflammation, or edema), (C) two crossing fibers 24, and (D) two crossing fibers 24 with an isotropic component 22. If fiber 20 of (A) is of interest and the target for a DTI measurement as demonstrated, the correct DTI result for the ideal fiber result 26. Nevertheless, the various mixed conditions result in misrepresentations 28, 30, and 32 of the targeted fiber, which is the major shortcoming of DTI.

To definitively resolve the issue regarding the utility of directional diffusivity in detecting white matter injury in MS and/or other CNS white matter disorders, a careful evaluation was performed on the mouse model of cuprizone intoxication that is widely employed to examine the mechanisms of CNS white matter de- and re-myelination. It has been demonstrated that axonal injury, inflammation, and demyelination co-exist at 4 weeks of continuous cuprizone feeding. Previous DTI studies showed that decreased λ_{∥} correlated with histology-confirmed axonal injury, while no significant increase of λ_{⊥} was seen, thus failing to reflect the concurrent demyelination. A Monte Carlo simulation modeling the three underlying pathologies was performed. Preliminary results suggested that the presence of infiltrating inflammatory cells exerted significant effect on the derived directional diffusivity reducing both λ_{∥} and λ_{⊥}, exaggerating the effect of axonal injury while diminishing the sensitivity to demyelination. This finding suggests that the current DTI analysis is suboptimal to accurately depict the underlying pathology in diseases with inflammation, such as MS.

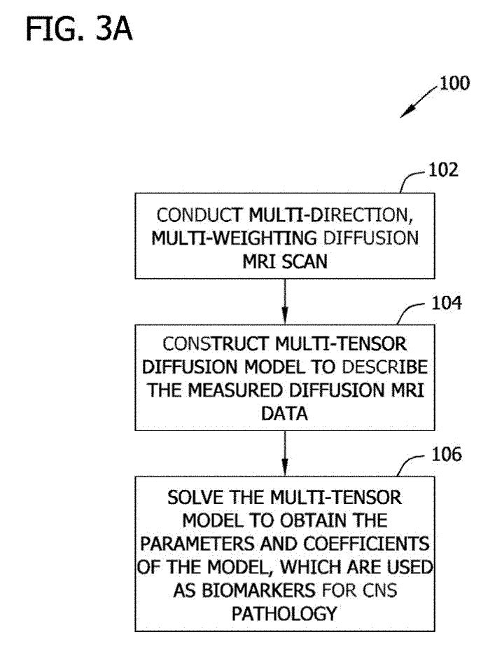

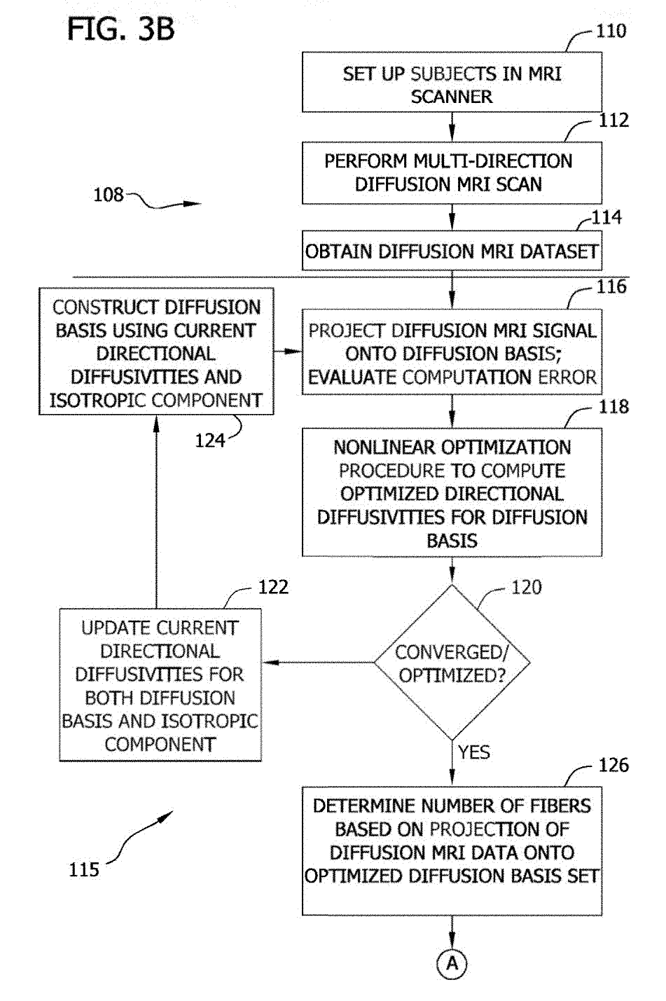

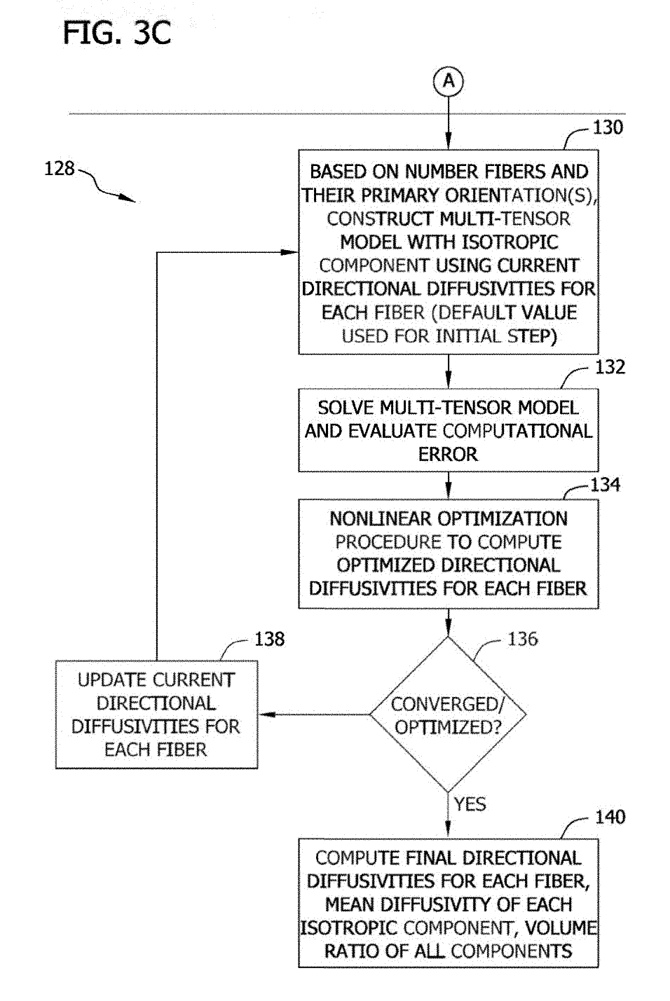

To address this shortcoming of DTI, a process allowing an accurate description of the underlying tissue pathology is described herein. FIG. 3A is a flow chart 100 illustrating the basic steps contemplated to detect and differentiate the underlying CNS white matter pathologies. First, a multi-direction, multi-weighting diffusion MRI scan is conducted 102 utilizing a signal acquisition and processing component. A multi-tensor diffusion model is constructed 104, and the multi-tensor model is solved 106 to obtain the parameters and coefficients of the model.

In the exemplary embodiment, a multiple-tensor based DBSI, or diffusivity component, is provided (FIGS. 3B and 3C). The method illustrated may be used to determine diffusivity of each diffusion tensor component within a tissue. In the multiple-tensor based DBSI, an MRI scan is performed 108. In performing the MRI scan, subjects are set up 110 in MRI scanner and a multi-direction diffusion MRI scan is performed 112. From the performed 112 MRI scan, a diffusion MRI dataset is obtained 114.

After an MRI scan is performed 108, number of fibers and their primary orientation is determined 115. In determining 115 the number of fibers and their primary orientation a diffusion MRI signal is projected 116 onto diffusion a basis and a computation error is evaluated. Next, a nonlinear optimization procedure is performed 118 to compute optimized directional diffusivities for diffusion basis. It is determined 120 whether the fibers are converged and optimized. If the fibers are determined 120 not to have been converged and optimized, the current directional diffusivities for both diffusion basis and isotropic components are updated 122. After update 122, a diffusion basis using current directional diffusivities and isotropic component is constructed 124 and projected 116 again. If the fibers are determined 120 to have been converged and optimized, the number of fibers based on projection of diffusion MRI data onto optimized diffusion basis set is determined 126.

After the number of fibers and their primary orientation is determined 115 (FIG. 3C), diffusivities of each fiber and isotropic components are determined 128. In determining 128 the diffusivities of each fiber and isotropic components, a multi-tensor model with isotropic component using current directional diffusivities for each fiber is constructed 130. A multi-tensor model is solved 132 and evaluated for computational error. Next, a nonlinear optimization procedure is performed 134 to compute optimized directional diffusivities for each fiber. It is determined 136 whether the fibers are converged and optimized. If the fibers are determined 136 not to have been converged and optimized, the current directional diffusivities for each fiber are updated 138 and the multi-tensor model is constructed 130 again. If the fibers are determined 136 to have been converged and optimized, a final directional diffusivity for each fiber is computed 140. Additionally, a mean diffusivity of each isotropic component, and a volume ratio of all components is computed 140.

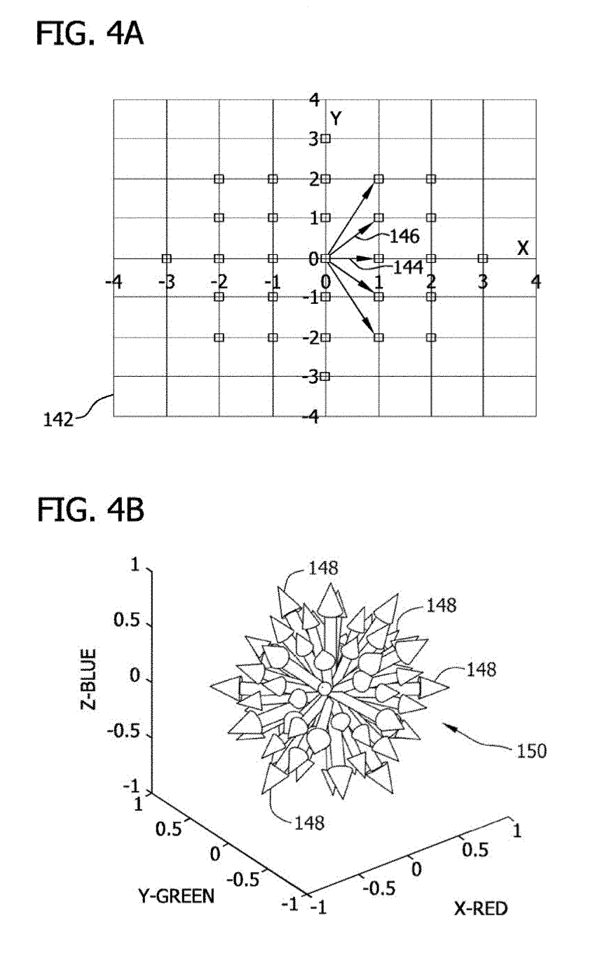

FIG. 4A-4B are illustrations of the design of an exemplary 99-direction diffusion-weighting scheme. As shown in the 2D schematic 142 in FIG. 4A, each diffusion-weighting direction is selected based on the grid point location. For example, the first diffusion weighting direction 144 is from origin (0, 0) to grid point (1, 0), the second diffusion weighting direction 146 is from (0, 0) to (1, 1), and so on. In this example, 99 diffusion directions are selected based on the 3D grid locations 148 shown by 3D model 150 in FIG. 4B.

An advantage of designing the 99-direction diffusion weighting gradients 148 based on regular grid locations is that the directions are uniformly sampled in the 3D space. No matter which direction the real axonal fiber orients, the scheme has no bias to it. Another advantage is that the weighting of diffusion gradients is naturally set as different values in this grid-based design, which is favorable in terms of determining multiple isotropic diffusion components.

However, embodiments described herein are not limited to this particular design. Any diffusion-weighting scheme that samples the whole 3D space uniformly and provides multiple weighting factors may work well resolving multiple-tensor reflecting the CNS white matter pathology.

Similar to diffusion basis function decomposition (DBFD), DBSI employs the following multi-tensor model as the first-step analysis:

$${S}_{k} = {\sum }_{i = 1}^{N} {s}_{i} exp ( - \vec{{b}_{k}} \cdot {λ}_{⊥} ) exp [ - \vec{{b}_{k}} \cdot ( {λ}_{∥} - {λ}_{⊥} ) * {cos}^{2} ( {θ}_{i} ) ] , \\ k = 1 , 2 , … 99 \qquad ( Equation 1 )$$

In Equation 1, N the number of diffusion basis components uniformly distributed in 3D space; \vec{b}ₖ is k^{th} diffusion gradient (k=1, 2, . . . , 99); λ_{∥} is the axial diffusivity and λ_{⊥} is the radial diffusivity; Sₖ is the measured diffusion weighted signal at direction \vec{b}ₖ; θᵢ is the angle between the diffusion gradient bₖ and the principal direction of the i^{th} diffusion basis.

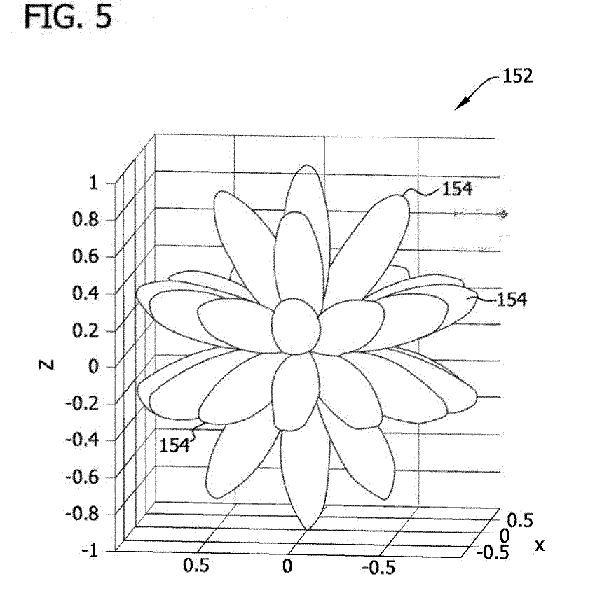

FIG. 5 illustrates a diffusion basis set 152 with 40 diffusion bases 154. As shown in FIG. 5, each diffusion basis 154 represents a candidate fiber orientation, and the diffusion basis 154 set is uniformly distributed in the 3D space. As described by Equation 1, the real fiber is treated as the linear combination of the entire diffusion basis set.

Instead of presetting λ_{∥} and λ_{⊥} at fixed values for the entire diffusion basis in DBFD, DBSI performs a nonlinear searching to estimate the optimal values of λ_{∥} and λ_{⊥} best fitting the acquired diffusion weighted data.

$$f ( {λ}_{∥} , {λ}_{⊥} , d ) = min ‖ {\sum }_{k = 1}^{99} \{ {S}_{k} - {\sum }_{i = 1}^{N} {S}_{i} {e}^{- \vert {\vec{b}}_{k} \vert \cdot {λ}_{⊥}} {e}^{- \vert {\vec{b}}_{k} \vert \cdot ( {λ}_{∥} - {λ}_{⊥} ) {cos}^{2} {θ}_{i}} - {{S}_{N + 1} {e}^{- \vert {\vec{b}}_{k} \vert \cdot d} \}}^{2} ‖ \qquad ( Equation 2 )$$

In Equation 2, Sᵢ (i=1, 2, . . . , N+1)≧0, λ_{∥} and λ_{⊥} are directional diffusivities, and d is the diffusivity of isotropic diffusion component with d, λ_{∥}, and ∥_{⊥} selected as the optimization variables. Unknown coefficients Sᵢ (i=1, 2. . . N+1) are not optimization variables because Sᵢ are not independent to ∥_{∥} or ∥_. Each Sᵢ is computed using the least square estimation under the nonnegative constraint (Sᵢ≧0) and the basic principle of sparsity as employed in DBFD during the nonlinear optimization procedure. After the optimization, the number of fibers and their primary axis directions are estimated similar to DBFD.

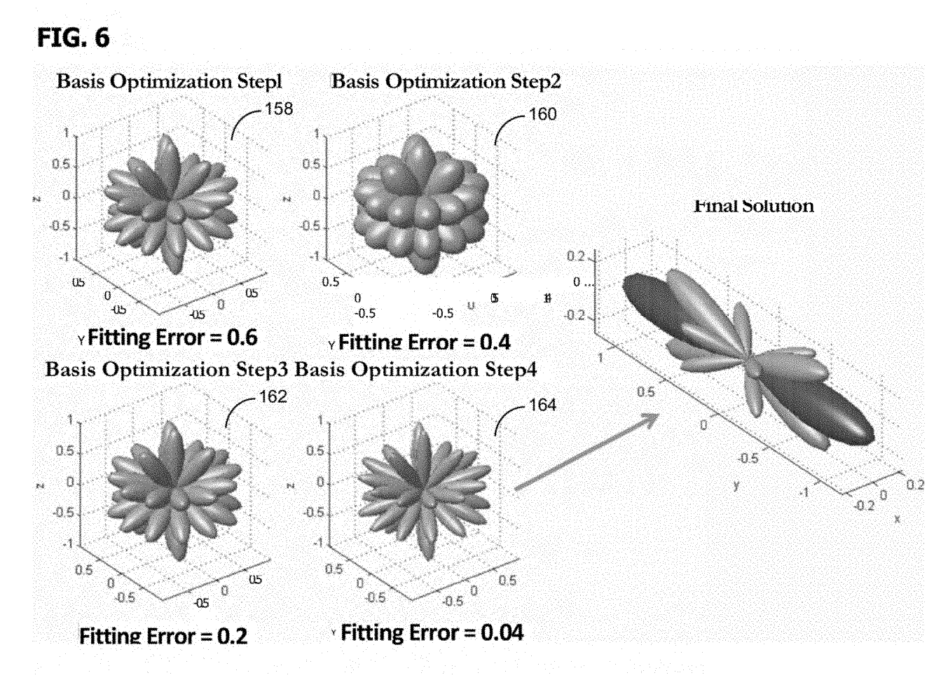

A unique feature of this disclosure is that the shape of each diffusion basis is not prefixed as in DBFD method. Instead, the basis shape is optimized during the optimization process to estimate both ∥_{∥} and ∥_{⊥}. This optimization process is demonstrated in FIG. 6 using a single axonal fiber 156 as the example. In the exemplary embodiment, experimental data is fitted by the linear combination of a diffusion basis set 154 in FIG. 5 with fitting error improved through iterations 158, 160, 162, and 164 until the optimal coefficients of linear combination of diffusion basis are estimated 166. In the exemplar embodiment, iteration 158 has a fitting error of 0.6, iteration 160 has a fitting error of 0.4, iteration 162 has a fitting error of 0.2, and iteration 164 has a fitting error of 0.04. An isotropic component was also considered according to Equation 2 in this process (not shown) to improve the optimization accuracy.

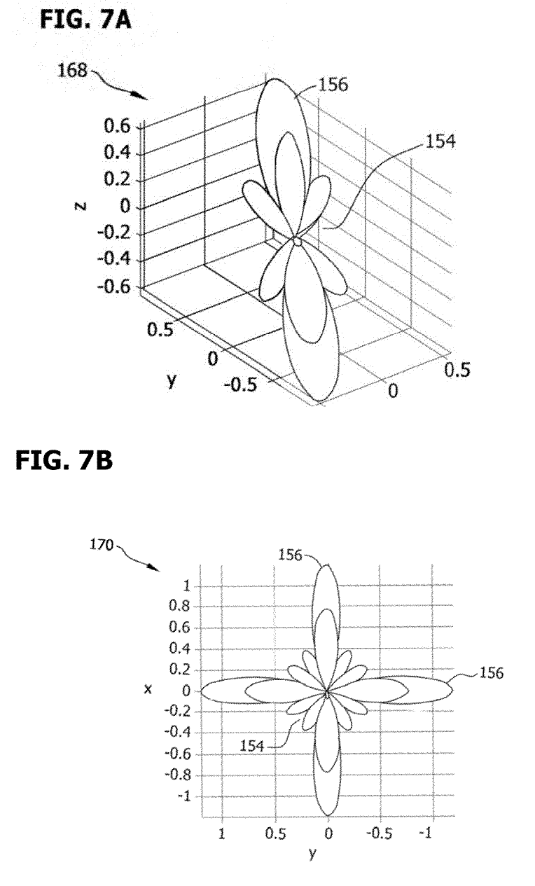

As shown in FIGS. 7A-7B, the diffusion basis 154 with direction close to that of the axonal fiber 156 contributes more significantly to the linear combination with higher magnitude of the coefficients Si. The diffusion basis 154 with direction away from that of the axonal fiber 156 has limited contribution to the coefficient of linear combination of the basis set fitting the experimental data. Both single 168 and two-fiber 170 tracts are demonstrated.

DBSI determines the number and primary direction of fibers according to the description of Equation 1. Each coefficient is associated with one diffusion tensor basis at a particular direction. These preliminary coefficients are grouped based on the magnitude and the closeness in orientations of the associated basis diffusion tensor. Coefficients smaller than a threshold determined by raw signal SNR are ignored. Significant coefficients with closely oriented (within 15 degrees) diffusion basis tensors are grouped as one fiber. The threshold of 15 degrees is set based on the desired angular resolution. Once the grouping process is complete, the averaged direction of the grouped diffusion basis is defined as the primary direction of the fiber.

Based on the number of fiber (anisotropic tensor) components and associated primary directions, DBSI constructs another multi-tensor model with the assumption of axial symmetry. A set of isotropic tensor components are included in the model:

$${S}_{k} = {\sum }_{i = 1}^{L} {S}_{i} {e}^{- \vec{\vert {b}_{k} \vert} \cdot {λ}_{{⊥}_{i}}} {e}^{- \vec{\vert {b}_{k} \vert} \cdot ( {λ}_{∥ \_ i} - {λ}_{{⊥}_{i}} ) \cdot {cos}^{2} {θ}_{i}} + {\sum }_{j = 1}^{M} {S}_{L + j} \cdot {e}^{- ( \vec{\vert {b}_{k} \vert} \cdot {d}_{j} )} \qquad ( Equation 3 )$$

In Equation 3, Sₖ is the measured diffusion weighted signal at diffusion gradient direction \vec{bₖ}. L is the number of estimated fibers in the imaging voxel. λ_{∥}_{_{—1}} and ∥_{⊥i} (i=1, 2, . . . , L) are the axial and radial diffusivity of the ith fiber. φᵢ is the angle between the diffusion gradient \vec{bₖ} and the primary direction of ith estimated fiber. dⱼ(j=1, . . . , M) are the diffusivities of M isotropic diffusion components. Sᵢ (i=1, 2. . . L) are fiber volume ratios and Sᵢ (i+L+1, L+2, . . . , L+M) are the volume ratio of isotropic components.

Based on this multi-tensor model, a nonlinear optimization search is constructed as following:

$$\{ Equation 4 )$$

$$h ( {λ}_{∥ \_ i} , {λ}_{⊥ \_ i} , i = 1 … L ) = min ‖ {\sum }_{k = 1}^{99} \{ {S}_{k} - {\sum }_{i = 1}^{L} {S}_{i} exp ( - {\vec{b}}_{k} \cdot {λ}_{⊥ \_ i} ) exp ( - {\vec{b}}_{k} \cdot {λ}_{∥ \_ i} - {λ}_{⊥ \_ i} ) {cos}^{2} ( {∅}_{i} ) ) - {{\sum }_{j = 1}^{M} {S}_{L + j} exp ( {\vec{b}}_{k} \cdot {d}_{j} ) \}}^{2} ‖$$

Equation 4 is subject to Sᵢ (i=1, 2, . . . , L+M)≧0. In this optimization procedure, isotropic diffusivity dⱼ (j−1. . . M) are not selected as optimization variables to reduce the total number of the free variables. Instead, isotropic diffusivities are uniformly preset within the physiological range. Directional diffusivities, λ_{∥}_{_}ᵢ and ∥_{⊥i} (i=1. . . L) of each anisotropic component are the only free variables to be optimized based on the experimental data and Equation 4 with the nonnegative constraint (Sᵢ≧0). All diffusion tensor's signal ratios Sᵢ (i=1, 2. . . L+M) based on T2-weighted (i.e., non-diffusion weighted) image intensity are computed with least square fitting during the nonlinear optimization procedure.

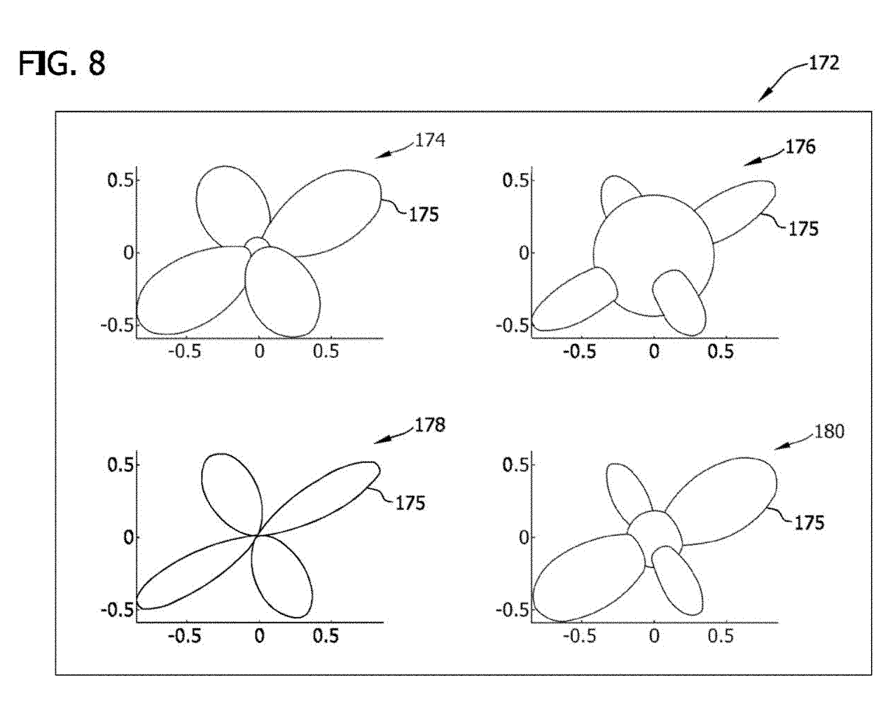

An optimization process 170, as shown in FIG. 8, is used to search the best directional diffusivities for each candidate fiber and compute all the volume ratios of each diffusion component. Process 170 demonstrates two crossing fibers (L=2). In such an embodiment, a first optimization 174 includes candidate fibers 175 with a fitting error of 0.4. Likewise, a second optimization 176 includes candidate fibers 175 with a fitting error of 0.2, a third optimization 178 includes candidate fibers 175 with a fitting error of 0.1, and a fourth optimization 180 includes candidate fibers 175 with a fitting error of 0.02

After the fourth optimization 180, the fitting error is smaller than 2%, which falls within the acceptable range. Therefore, the directional diffusivity of each candidate fiber 175, and corresponding volume ratios computed after the optimization 180 are determined as the final DBSI results. In the DBSI algorithm, the nonlinear optimization procedure is executed based on criteria including maximal iteration numbers, tolerance of mesh size, tolerance of variable, tolerance of function, accepted accuracy, and many other criteria set according to the need. Once some or all of these criteria are met according to the preset level, the optimization procedure is considered satisfactorily fit the data and the optimization stops.

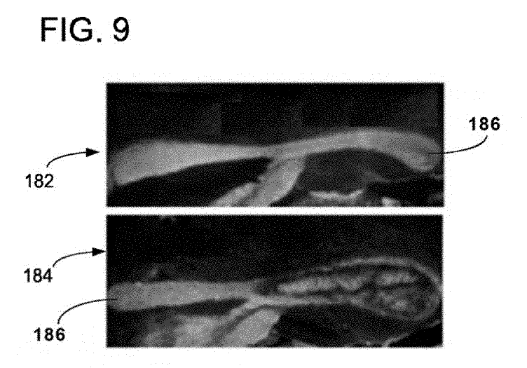

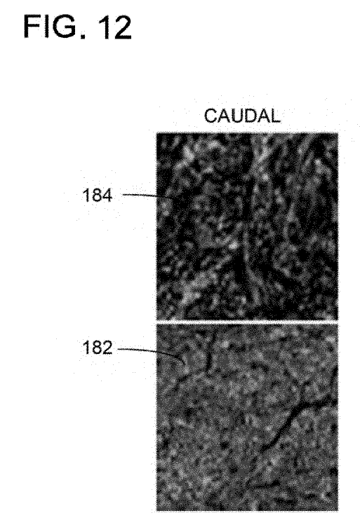

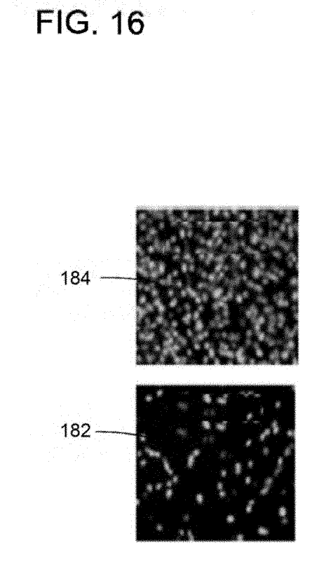

To determine the capability of the newly developed DBSI approach in detecting and differentiating the underlying co-existing pathology, the cuprizone model was again employed to compare conventional DTI with the new DBSI analysis. Striking contrast between DTI and DBSI was observed at the corpus callosum from C57BL/6 mice treated with cuprizone for 4 weeks. DTI failed to detect demyelination and overestimated axonal injury even with 99-direction diffusion weighting, while offering no information on inflammation. However, DBSI correctly reflected the presence of demyelination (FIG. 9, MBP), axonal injury (FIG. 12, SMI-31), and inflammation (FIG. 16, DAPI).

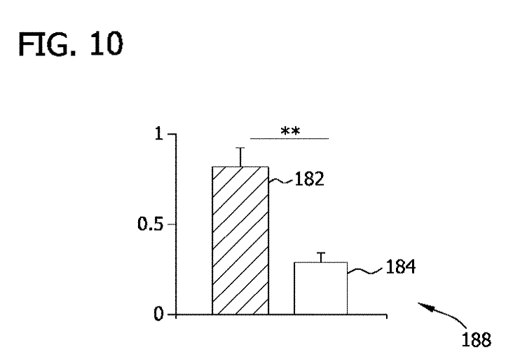

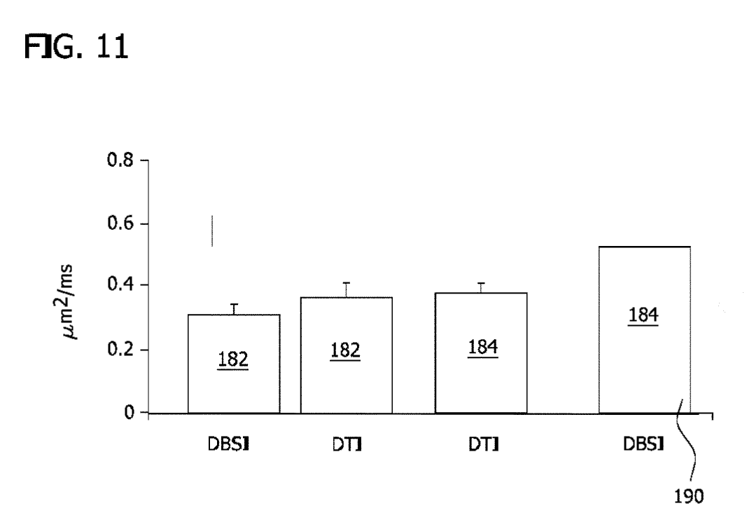

FIG. 9 is an illustration of a sagittal view of corpus callosum from a control 182 and a 4-week cuprizone fed male C57BL/6 mice (n=5) 184 examined using DBSI and DTI. As shown by myelin basic protein 186 immunostaining, significant demyelination in the caudal corpus callosum is seen by reduced MBP-positive area 188 (FIG. 10) and increased radial diffusivity 190 (FIG. 11) derived using DBSI. Consistent with previous reports, lack of increase in DTI derived radial diffusivity failed to reflect the histological finding of demyelination (FIG. 11).

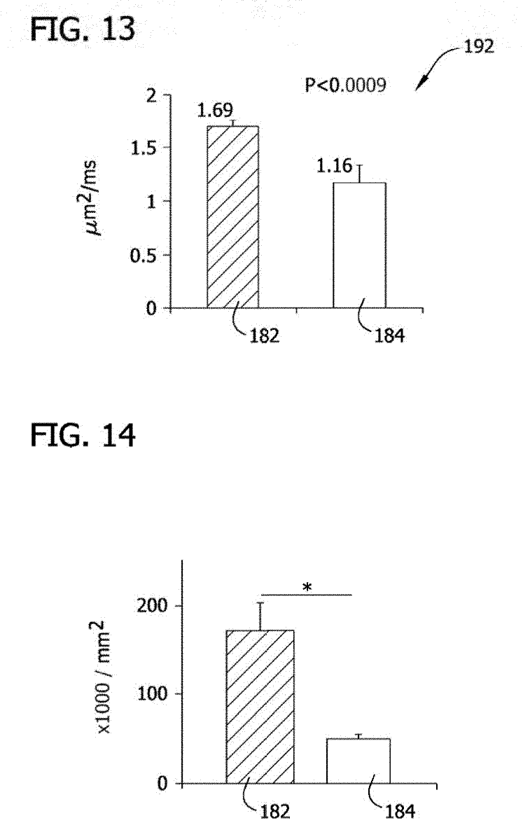

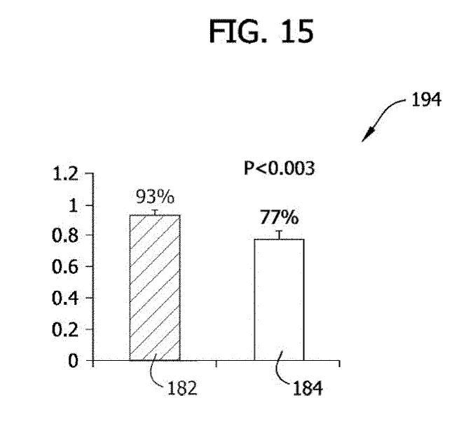

FIG. 12 illustrates that similar to previous findings that decreased SMI-31 staining seen in corpus callosum from 4-week treated mice 184 (n=5, −43%) from control 182. DBSI derived axial diffusivity 184 (FIG. 13) decreased (−31% from the control 182) to reflect the histology proved axonal injury (FIG. 14). The axonal fiber tract density 194 (FIG. 15) was also derived using DBSI expressing as volume ratio. Due to the infiltrating inflammatory cells, the density of axonal fiber tracts was reduced from 93% to 77%, a finding not available for conventional DTI.

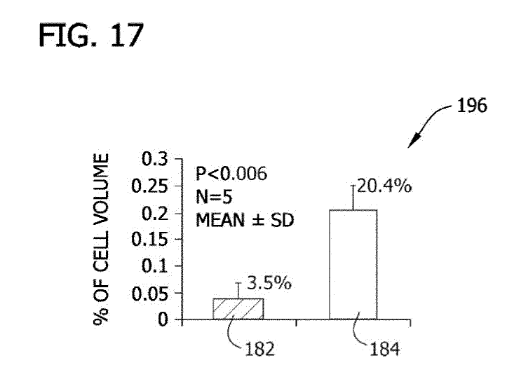

FIG. 16 illustrates that similar to previous findings that increased DAPI staining seen in corpus callosum from 4-week treated mice 184 (n=5, 16543±3523 counts/mm²) comparing with that of the control 182 (n=5, 3473±582 counts/mm²). Inflammatory cell infiltration 196 derived using DBSI is 20% of total volume in 4-week cuprizone treated corpus callosum 184, above the baseline 3.5% cellular content from control 182 in FIG. 17. This is consistent with the significantly increased DAPI positive stains in the same region; information has not been available using DTI.

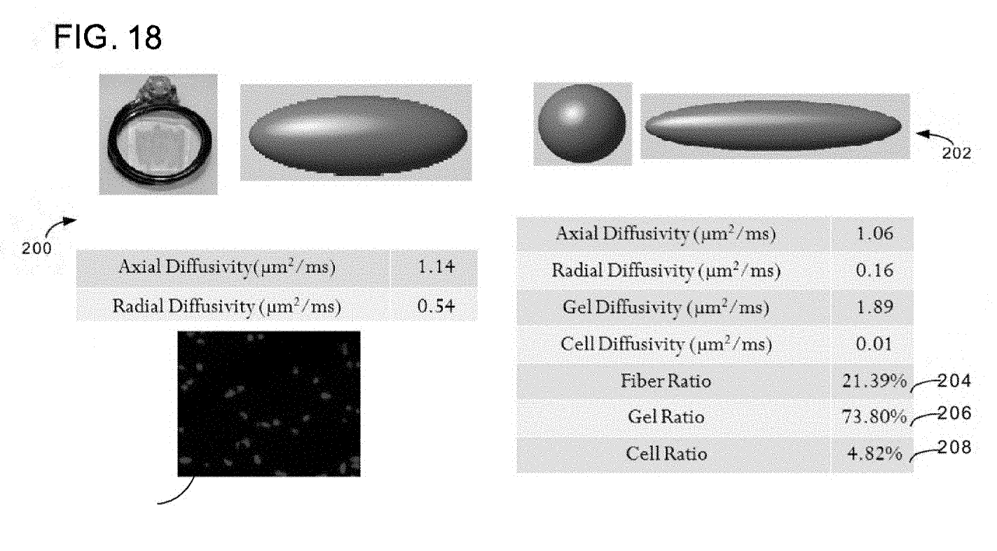

In another embodiment, 99-direction diffusion weighted images are analyzed following one or more operations described above to determine the number of intravoxel fibers and isotropic components on a laboratory fabricated phantom containing mouse trigeminal nerves with known in vivo DTI character and isotropic gel as shown in FIG. 18.

Diffusion weighted MRI was performed on the phantom using 99 distinct diffusion weighting gradients for both DTI 200 and DBSI 202 analysis. For the pure gel, DTI 200 and DBSI 202 estimated the isotropic apparent diffusion coefficient to be identical at 1.91 μm²/ms suggesting both methods are accurate for simple medium. When examining the mixture of fiber/gel in this phantom using DTI 202, the isotropic gel component was not identified. In addition, the true fiber diffusion anisotropy (FA=0.82±0.005) determined previously using an in vivo high resolution DTI was not obtained. In contrast, using the newly proposed DBSI identified a fiber ratio 204 of 21%, a gel ratio 206 of 74%, and a cell ratio 208 of 5% with correct fiber diffusion anisotropy of FA=0.83. The anisotropy was compared because it was previously observed that diffusion anisotropy is preserved in vivo and ex vivo in mouse nerve fibers.

Methods described herein facilitate determination of an axial diffusivity, a radial diffusivity, and/or a volume ratio of a scanned volume of tissue with increased accuracy relative to known methods, which are distinguishable at least as follows.

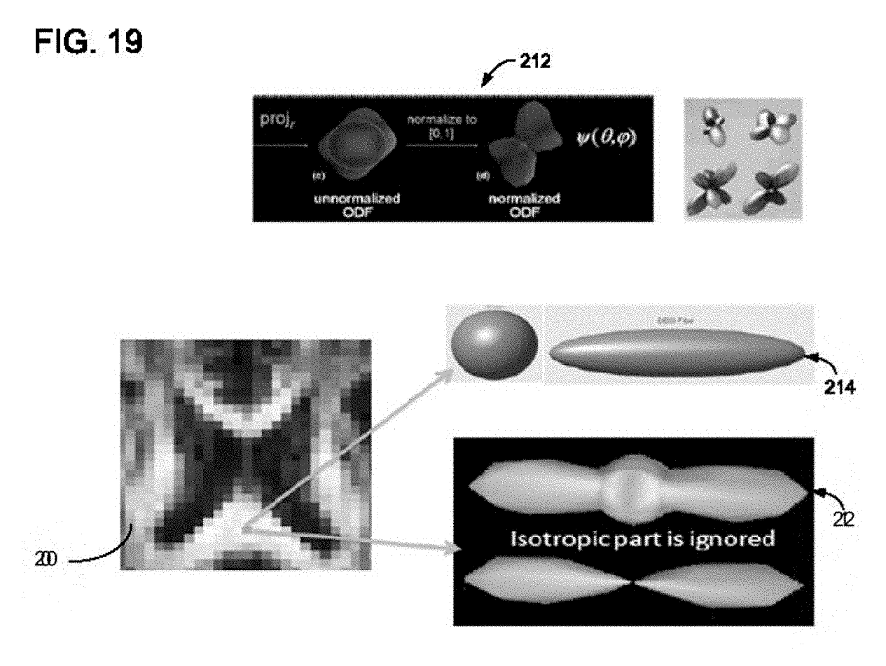

FIG. 19 is a comparison of diffusion spectrum imaging (DSI) 212 and DBSI 214 from human subjects 200. DSI 212 is a method that attempts to directly measure the probability distribution function of the displacement of water molecules without an assumption of tissue structure or the shape of probability distribution function. It was proposed to identify multiple fibers within an image voxel. The use of orientation distribution function (ODF) by DSI effectively estimates angles of crossing fibers. However, its ODF based analysis does not offer other crucial quantitative information of water diffusion relevant to tissue physiology and pathology such as the apparent diffusion coefficients, diffusion anisotropy, or the volume ratio of different components. Therefore, DSI's applications are limited to fiber tracking.

The presence of an isotropic component within the image voxel is an important biomarker for cell infiltration, edema, and tissue loss. As shown in FIG. 19, the isotropic diffusion component is ignored in DSI 212 operation for the better estimation of the fiber orientation. In contrast, DBSI 214 quantitatively separates the isotropic from fiber component with accurate isotropic diffusivity assessment.

Operationally, DSI requires high diffusion weighting gradients of various magnitudes and directions to accurately estimate the ODF, a typically impractical challenge on regular clinical MR scanners. In contrast, DBSI facilitates operation with the clinically used diffusion weighting gradient strength and smaller number of directions. Thus, DBSI may be performed on clinical MR scanners with typical hardware resources.

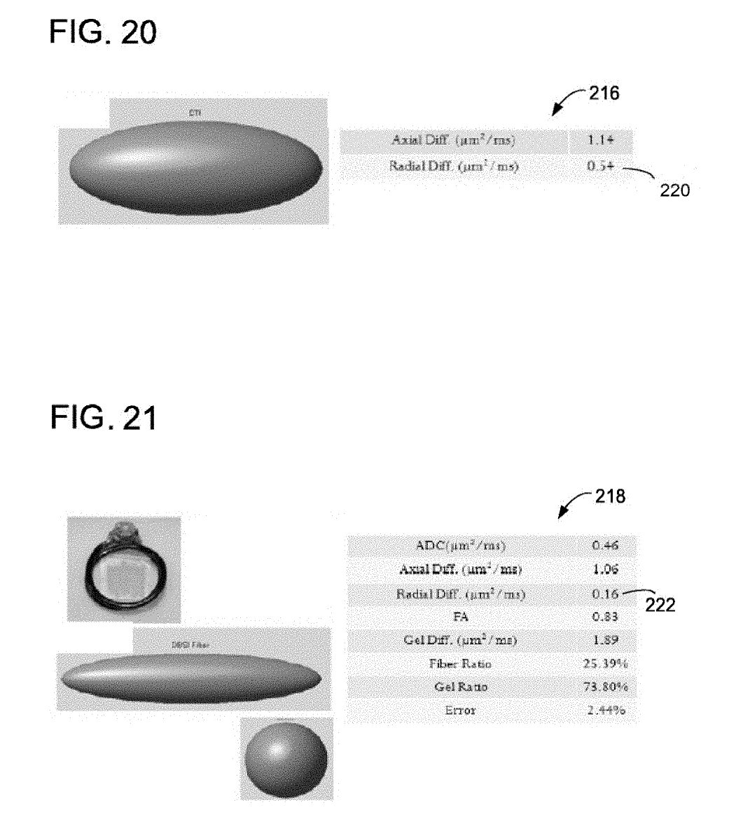

FIG. 20 is a diffusion tensor imaging (DTI) 216 for mouse trigeminal nerve embedded in gel, and FIG. 21 is a DBSI 218 for mouse trigeminal nerve embedded in gel. DTI 216 derived radial diffusivity is very dependent on the tissue environment, and inaccurate assessment is common due to both the intra- and inter-voxel partial volume effect as demonstrated in FIG. 20. Using a simple yet realistic phantom constructed from fixed mouse trigeminal nerves and gel, as described above and as shown in FIG. 21, DTI 216 significantly over estimated the radial diffusivity 220, while DBSI 218 correctly quantified diffusivities 222, anisotropy, and volume ratios of all components.

This phantom study demonstrates the superior results enabled by DBSI in quantifying the overwhelming isotropic component within the image voxel and reporting correct diffusion properties of both the fiber and its environment. Embodiments described herein facilitate correctly estimating the extent of axonal loss noninvasively (e.g., in a clinical setting).

In one embodiment, eight trigeminal nerves from 4 normal male C57BL/6 mice were isolated after fixation. Diffusion MR spectroscopy was performed at 19° C. using a custom-built surface coil with the following parameters (common to all nerve fiber measurements): max b=3200 (s/mm²), repetition time (TR) 2 s, echo time (TE) 49 ms, time between application of gradient pulses (A) 20 ms, duration of diffusion gradient on time (δ) 8 ms, number of averages 4, 99-direction diffusion weighting gradients. Three diffusion tensor components were observed: anisotropic diffusion (75.9±2.6%: axon fibers), restricted isotropic diffusion (12.1±0.99%: cells), and non-restricted isotropic diffusion (12.1±2.5%: extra- axonal and extracellular water). The assignment of cell and water components was based on the DBSI-derived spectrum of isotropic diffusion.

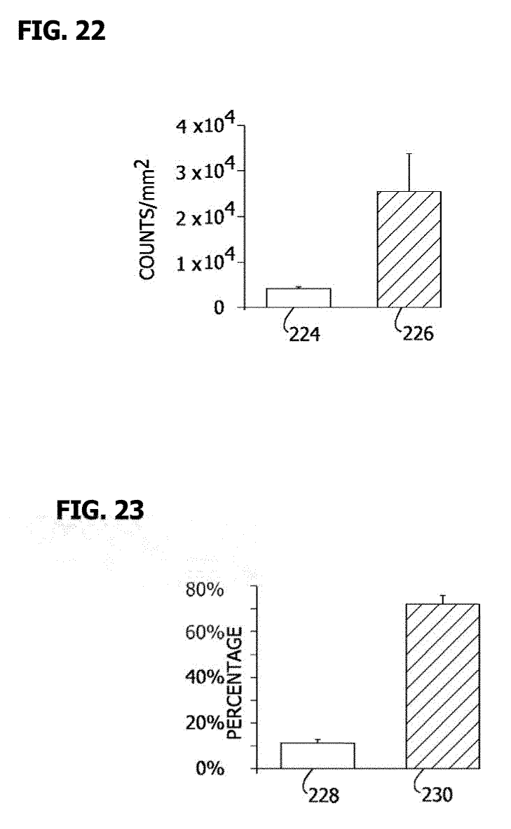

FIG. 22 illustrates a DAPI 224 and SMI-31 226 staining counts of fixed mouse trigeminal nerves. In such an embodiment, nucleus and axon staining was performed using 4′,6′-diamidino-2-phenylindole (DAPI) and phosphorylated neurofilament (SMI-31) to count cells (4109±629/mm²) and axons (25434±8505/mm²). The fiber 228 to cell 230 ratio estimated by DBSI is comparable to that estimated by SMI-31 and DAPI count ratio (FIG. 23).

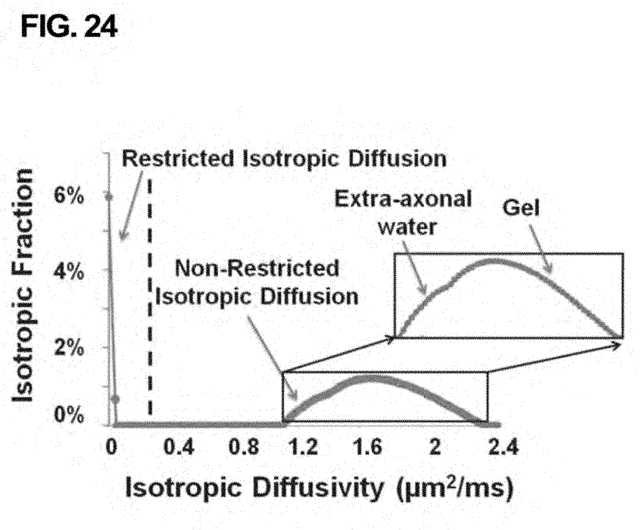

Five fiber-gel samples were examined at 19° C. using DBSI to quantify anisotropic and isotropic diffusion, and T2W MRI to quantify total gel signal intensity (FIG. 24). The powder-average effect of the 25% (FIG. 24) isotropic diffusion component in the fixed trigeminal nerve is apparent when comparing λ_{∥}and λ₁₉₅ derived using DBSI (λ_{∥}=1.07±0.05 μm²/ms; λ_{⊥}=0.12±0.01 μm²/ms) vs. DTI (λ_{λ}=0.77±0.03 μm²/ms; λ_{⊥}=0.17±0.02 μm²/ms). Compared to DBSI, DTI underestimated λ_{∥} by 28%, while overestimating ∥_{⊥} by 42%.

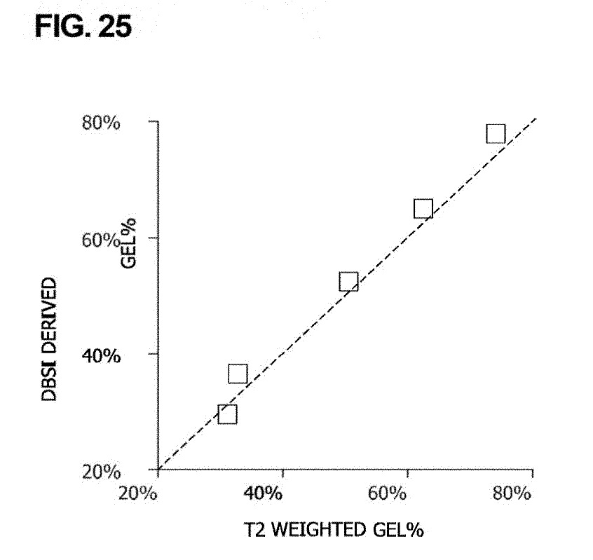

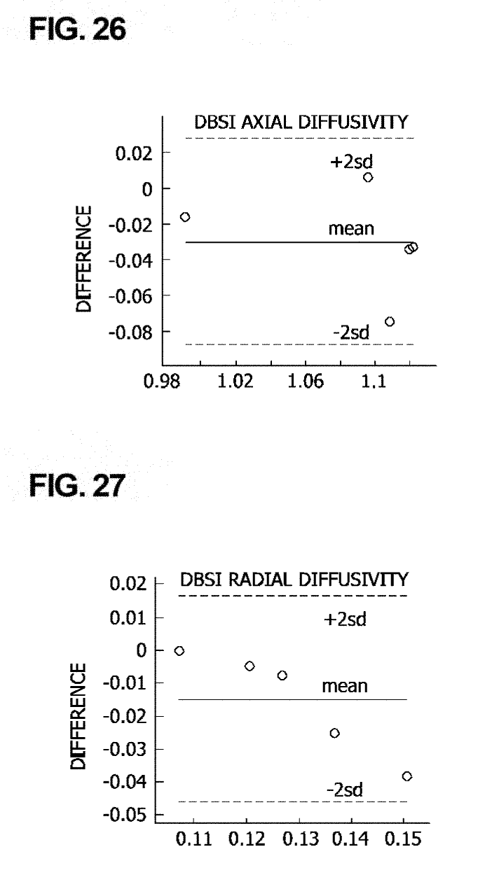

The DBSI-determined gel water fraction closely matches that determined using T2W MRI as shown in FIG. 25, suggesting the potential of DBSI to estimate edematous water from more freely diffusing water in regions of tissue loss. The comparable λ_{∥} (FIG. 26), λ_{⊥} (FIG. 27) derived from trigeminal nerves with and without gel was confirmed by Bland-Altman plots, indicating that DBSI can correctly assess fiber diffusion properties in the presence of edema or tissue loss.

FIG. 28 is an illustration of six fixed trigeminal nerves grouped into three pairs of crossing fibers at 32°, 58°, and 91° juxtaposed with 2% agarose gel. DBSI-estimated crossing fiber angles 300 compare favorably with those derived using an orientation distribution function (ODF) by DSI 302 and general q-sampling imaging (GQI) 304. DBSI-quantified mean fiber 300 λ_{∥}=1.14±0.06 μm²/ms, λ_{⊥}=0.12±0.02 μm²/ms agreed well with measured values for a single fiber without gel λ_{∥}=1.07±0.05 μm²/ms, λ_{⊥}=0.14±0.02 μm²/ms. For 91°, 58°, 32° phantoms, DBSI-derived gel percentages were 15%, 14%, and 50%, in close agreement with T2W MRI determined 18%, 13%, and 45%. DSI 302 and GQI 304 failed to resolve crossing fibers at 32° and 58° (FIG. 28).

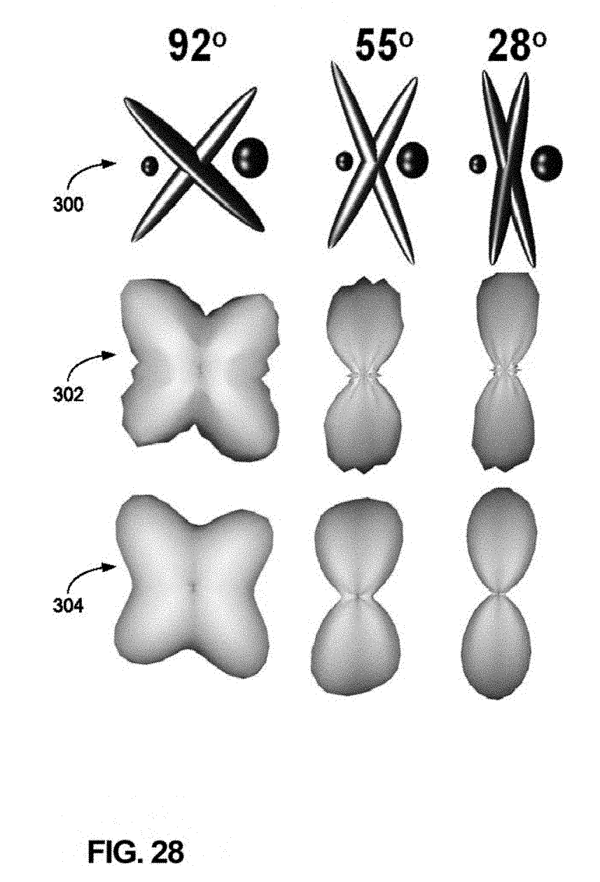

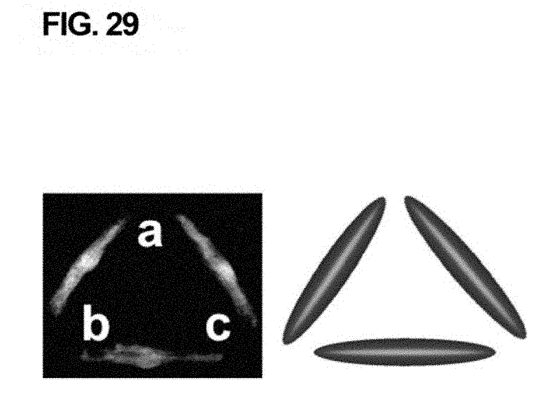

To further demonstrate the capability of DBSI to resolve multiple crossing fibers, a 3-fiber crossing phantom was built using fixed mouse trigeminal nerves arranged in an approximate equilateral triangle with inner angles of (a/b/c)=(75°/55°/50°, as is shown in FIG. 29.

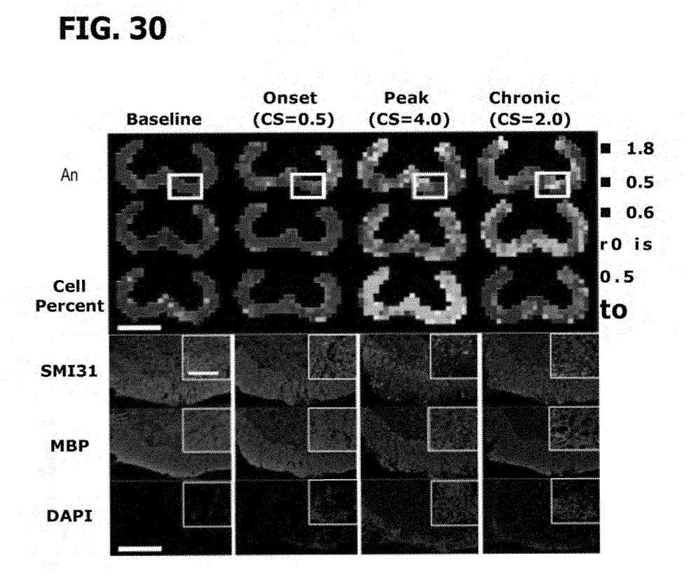

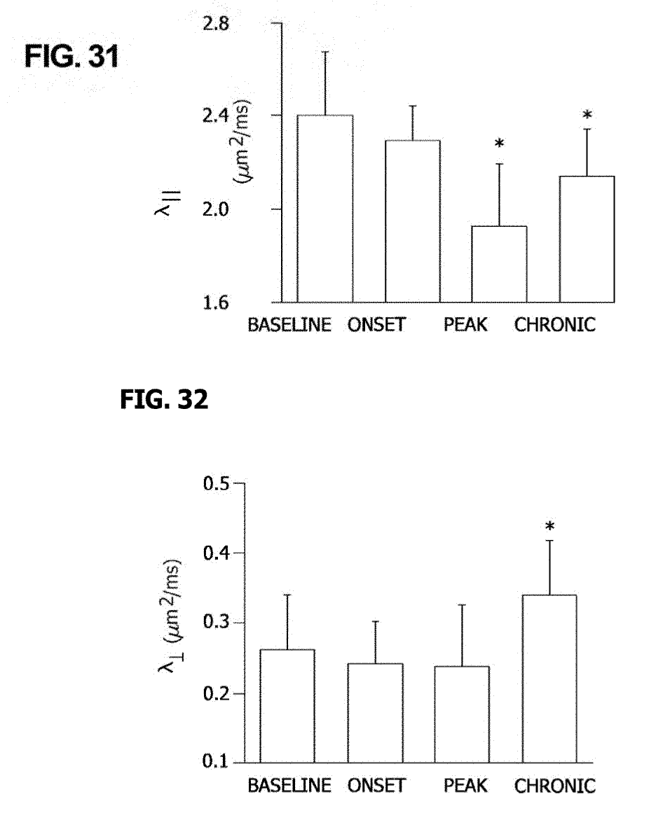

A cross-sectional study was performed on B6-EAE mice spinal cords at baseline (control), onset, peak, and chronic states, followed by IHC (N=5 for each time point). In the representative mouse, λ_{∥} decreased at the peak and recovered slightly at the chronic EAE stage, consistent with decreased SMI-31 staining followed by the recovery of the staining as is shown by FIGS. 30 and 31. Increased λ_{⊥} was seen at EAE peak and continued to increase to the chronic EAE stage, consistent with the MBP staining gradually losing its intensity as shown in FIGS. 30 and 32.

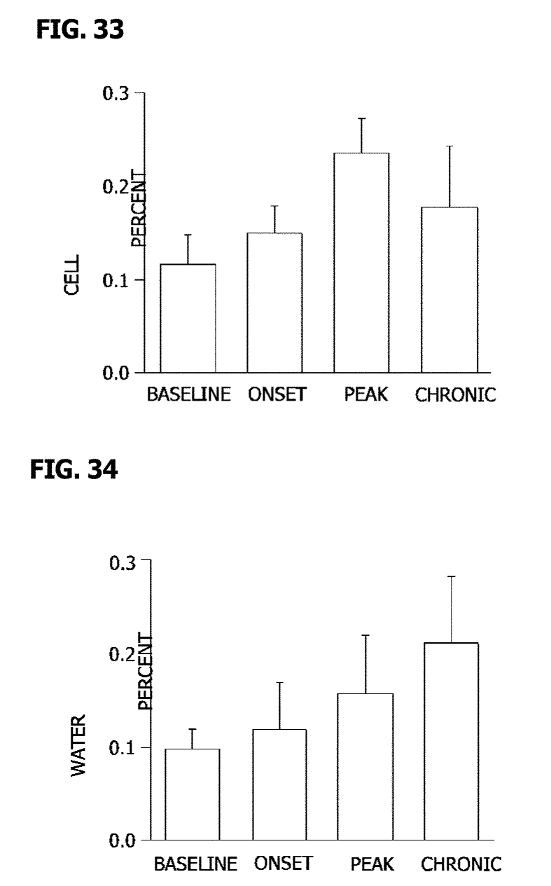

DBSI revealed cell infiltration at peak EAE, consistent with DAPI staining and clearly indicating the presence of inflammation (FIGS. 30 and 33). Quantitative analysis of the ventrolateral white matter DBSI parameters closely reflects the same pathology profile suggested by IHC shown in FIGS. 31-34. DBSI reflects axon and myelin injury more accurately than that previously determined by DTI, and correctly depicts inflammatory pathological features of the spinal cord white matter from EAE mice in terms of both cell infiltration and vasogenic edema as shown in FIGS. 33 and 34.

Spherical Harmonic Decomposition (SHD) has been proposed as a method for classifying imaging voxels into isotropic, single-, and multi-fiber components based on SHD coefficients. However, SHD cannot accurately estimate the intra-voxel fiber numbers, fiber volume fractions, fiber anisotropy, or fiber orientations. Even in the simple case of two fibers, it is not possible to use SHD to uniquely determine the intra-voxel fiber numbers and orientation since both the volume fraction and relative fiber orientations interfere with the higher order SHD components in a similar fashion. Similar to DSI, SHD also requires high diffusion weighting gradients. In contrast, DBSI facilitates separating and quantifying the isotropic and individual anisotropic (fiber) components while maintaining the use of low diffusion weighting gradient magnitudes.

Q-ball imaging of the human brain is a method closely related to DSI. In DSI, the ODF is reconstructed by sampling the diffusion signal on a Cartesian grid, Fourier transformation, followed by the radial projection. Q-ball imaging acquires the diffusion signal spherically and reconstructs the ODF directly on the sphere. The spherical inversion is accomplished with the reciprocal space funk radon transform (FRT), a transformation of spherical functions that maps one function of the sphere to another. Q-ball and DSI are theoretically equivalent and generate similar ODF. However, q-ball methods are not capable of estimating fiber angles as well as quantifying multiple tensor parameters.

Independent Component Analysis (ICA) has been proposed for application in DTI tractography to recover multiple fibers within a voxel. Although the angle of crossing fibers within voxels can be estimated to within 20 degrees of accuracy, eigenvalues cannot be recovered to obtain the complete tensor information such as the Fractional Anisotropy (FA).

Moreover, it has been proposed to use a high angular resolution diffusion imaging (HARDI) data set as a method that is capable of determining the orientation of intra-voxel multiple fibers. For example, up to 2-fiber components and one isotropic component may be considered. Similar to DBSI, HARDI methods have employed a mixed Gaussian model incorporating the isotropic diffusion component. However, HARDI is very different in nature compared with DBSI. For example, (i) HARDI fails in voxels with more than 2 fibers; (ii) HARDI does not work in voxels with more than 1 isotropic component, which is commonly seen in pathological conditions with both cell infiltration and edema; (iii) HARDI fails to compute isotropic diffusivity, improving fiber orientation estimation at the expense of removing the isotropic diffusion component; (iv) HARDI cannot compute the absolute axial and radial diffusivities for each component fiber; (v) HARDI cannot compute the true volume fractions of each fiber or isotropic component. In contrast, DBSI facilitates achieving all the goals enumerated above because it may be used to solve for issues that HARDI ignores or simplifies. HARDI-based methods have aimed to enhance the tools available for fiber tracking but do not compute the directional diffusivities of fibers, the isotropic diffusivity, or true volume fractions.

In summary, diffusion MRI methods in the field currently focus on determining the primary orientation of crossing fibers within one voxel. To achieve this goal, most have to relax the condition needed for accurate estimation of diffusivity or the volume ratio of individual component. DBSI facilitates not only resolving the primary direction of each fiber component, but also identifying and quantifying one or more other physical properties available from the diffusion measurements.

With the quantified fraction, axial diffusivity, and radial diffusivity of each fiber as well as the fraction and mean diffusivity of each isotropic diffusion tensor, CNS white matter pathology maps corresponding to the classic immunohistochemistry staining of excised tissues may be generated. For example, based on the axial diffusivity distribution intact (or injured) axonal fiber tract fraction may be estimated and the fraction distribution map may be generated to reflect the classic phosphorylated neurofilament (SMI-31, for intact axons), or dephosphorylated neurofilament (SMI-32, for injured axons), staining. The restricted isotropic diffusion component estimated using DBSI constitutes a map of cell distribution corresponding to nucleus counting using DAPI staining on the fixed tissue allowing a direct estimate the extent of inflammation in patient CNS white matter.

In the preceding discussion, a method has been developed incorporating the diffusion profile of each component within the image voxel to perform the tissue classification based on the raw diffusion MRI data. The typical classification is performed using the generated parameters, not the source data. This approach generates realistic “noninvasive histology” maps of various CNS white matter pathologies directly related to the actual immunohistochemistry staining that is only available after tissue excision and fixation. Although an accurate assessment of the underlying white matter pathologies may or may not correctly reflect clinical symptoms during the early phase of the disease, it would likely predict the long-term patient disability. Such a quantitative assessment of CNS white matter that tracks integrity would enable a clinically-based intervention for the patient. For example, current MS treatments follow a standard dosing regimen, with limited opportunity to adjust management for individual patient responses. By quantitatively distinguishing and tracking inflammation, and axon and myelin injury, DBSI provides the opportunity for efficient assessment of disease-modifying interventions and allows treatment planning to reflect individual patient response.

### Quantitative Diferentiation of Inflammation, Solid Tumors, and Nerve Damage in Human Diseases

In various aspects, the DBSI method disclosed herein above may be ideal to identify solid tumors in various organs, e.g., brain tumors, and prostate cancer (PCa). In the initial application of DBSI to detect brain tumors, it was observed that the apparent diffusion coefficient (ADC) profile differs among various types of brain tumors. The ADC profile for glioblastoma multiforme (GBM, WHO Grade IV) and primitive neuroectodermal tumor (PNET, WHO Grade IV) overlaps (0.8≦ADC≦1.6 μm²/ms) with but significantly differs from Anaplastic Ependymoma (WHO Grade III, 0.2≦ADC≦1.0 μm²/ms). The ADC profiles of live (0.8≦ADC≦1.6 μm²/ms) and dead (0.5≦ADC≦1.5 μm²/ms) GBM cells also overlap, yet are still different. Low-grade brain tumors (WHO Grade I or II) including optic nerve glioma, diffuse astrocytoma may also have much lower cellularity and lower ADC range (0≦ADC≦0.2 μm²/ms, overlapping with immune cells). Thus, a pre-determined ADC profile for one kind of brain tumor is unlikely to detect different kinds of tumors in brain or in other organs with the needed accuracy. The complication of detecting brain tumors also involves the accuracy of crossing fiber resolution.

Herein, DBSI was modified to enable a DBSI-based diagnostic method (Diffusion [MRI] Histology, i.e., D-Histo) configured to quantitatively differentiate prostate disorders characterized by histologically distinct regions within the prostate. In one aspect, D-Histo may be utilized to differentially diagnose at least several prostate disorders including, but not limited to, prostate cancer, prostatitis (prostate gland inflammation), and benign prostatic hyperplasia.

Although the D-Histo method, a modified DBSI-based diagnostic method, disclosed herein below is discussed in the context of quantitatively differentiating prostate disorders, it is to be understood that the D-Histo method may be modified for use in quantitatively differentiating any disorder characterized by quantifiable morphological changes from a normal/healthy state without limitation. Non-limiting examples of suitable disorders for quantitative differentiation using the D-Histo method disclosed herein include: bladder cancer, breast cancer, cervical cancer, pancreatic cancer, prostate cancer, myocarditis, myocardial infarction, myositis, and central/peripheral neuropathies. In addition, the D-Histo method is ideally suitable for assessing nerve function while simultaneously quantifying and differentiating nerve pathologies, allowing a direct correlation between nerve function and pathologies.

Prostate cancer (PCa) is the second most common cause of cancer-related death in American men. The first line of screening is performed during an annual physical through a digital rectal exam (DRE) and with a blood test to measure prostate specific antigen (PSA) level. The current standard imaging approach consists of detecting significant PCa, guiding biopsies, and active surveillance. Ultrasound-guided needle biopsy is the clinical standard for PCa diagnosis; however, this biopsy technique misses 20-30% of clinically significant tumors. Existing non-invasive diagnostic methods, such as multiparametric MRI (mpMRI) are associated with a significant false positive rate as a diagnostic tool, thus reducing the effectiveness of cancer detection. Multiparametric MRI mainly includes T1-weighted (T1W) imaging, T2-weighted (T2W) imaging, diffusion-weighted imaging (DWI), and Dynamic Contrast Enhanced (DCE) imaging. Many other prostate pathologies mimic the same signals and contrast as PCa, particularly in T2W and DWI images. False positive mpMRI results are largely due to heterogeneous signals of chronic inflammation, stromal benign prostatic hyperplasia (BPH), scarring, bleeding, infection, fibrosis, and glandular atrophy that may be falsely interpreted as PCa.

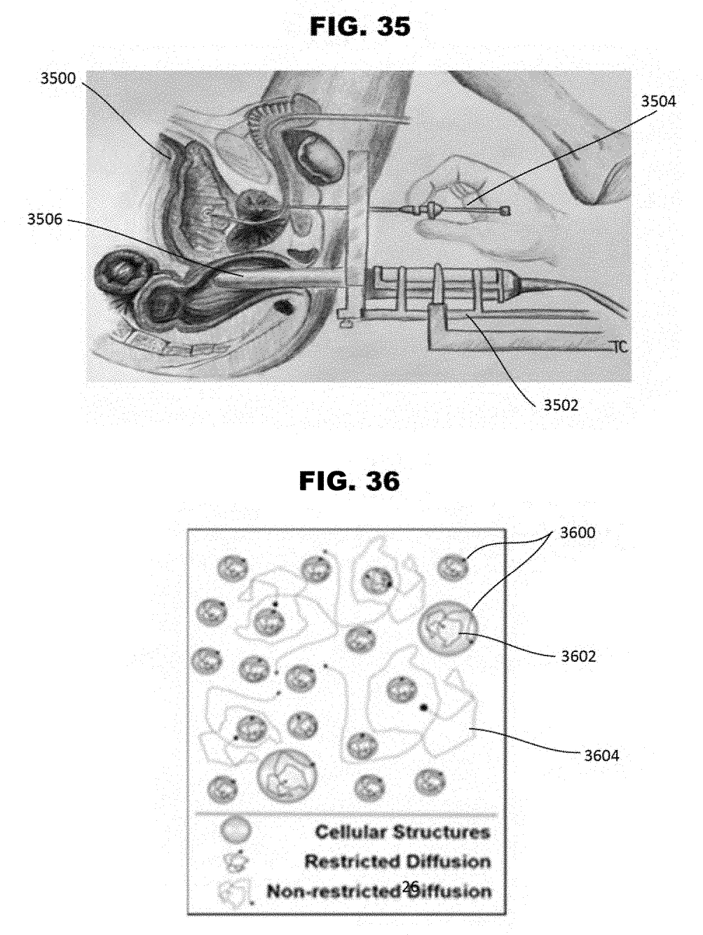

FIG. 35 shows the components of a targeted prostate cancer biopsy procedure for a patient 3500. A perineal template 3502 allows a biopsy needle 3504 to be guided into the prostate of the patient 3500. In this example, the needle 3504 is an 18 gauge needle (1.3 mm diameter) and used for seed placement. The perineal template 3502 includes an ultra sound probe inserted in the rectum for needle guidance.

Without being limited to any particular theory, microscopic barriers in the body (e.g., cellular and nuclear membranes) constrain the free Brownian motion of water molecules, resulting in a reduced apparent diffusivity measurable by diffusion MRI. Within the diffusion time range achievable in most clinical MRI scanners, water molecules inside cellular structures (spheres 3600 in FIG. 36) experience highly restricted diffusion (solid lines in 3602 FIG. 36), leading to a small apparent diffusion coefficient (ADC). Extracellular water has faster, non-restricted diffusion (dashed lines 3604 in FIG. 36), leading to a relatively large ADC. Raw diffusion MRI signals are a mixture of different types of constrained water diffusion, carrying rich microstructural cell information.

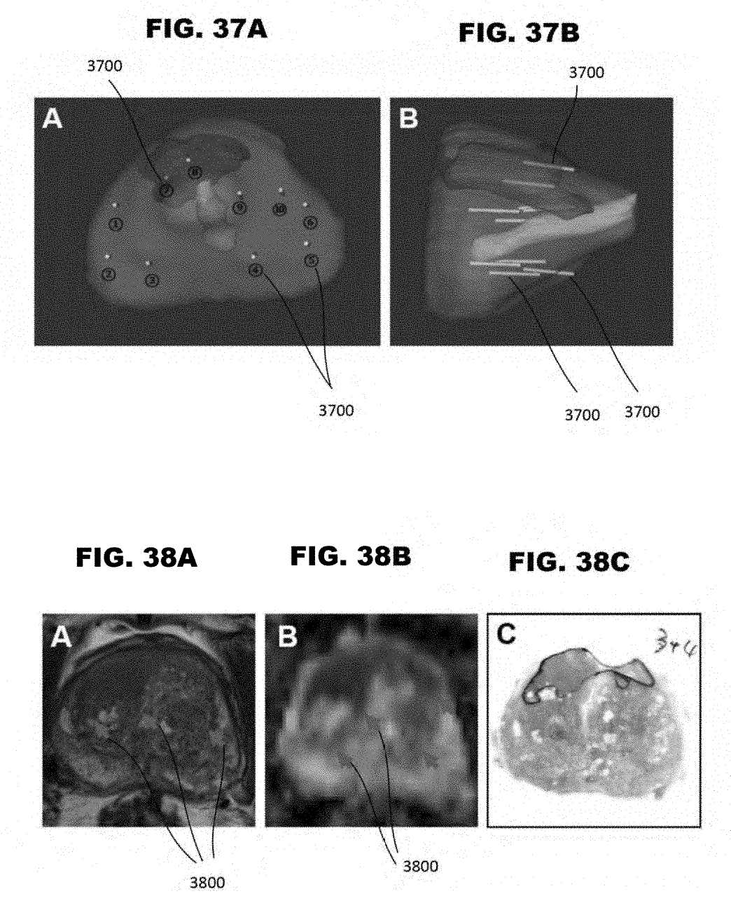

FIG. 37A shows a 3-D reconstruction of the prostate in a transverse view using mpMRI, as used for biopsy guidance purposes and FIG. 37B is a 3-D reconstruction of a prostate in a sagittal view. Needle biopsy locations 3700 are shown in FIGS. 37A-37B. As may be shown in FIGS. 37A-37B, numerous biopsy needles 3700 are commonly used. Improved accuracy in PCa detection via the D-Histo method as explained below can save biopsy patients from extra biopsy needles, such as those currently required outside the suspicious tumor foci.

In various embodiments, a modification of DBSI termed D-Histo is used to detect and distinguish PCa, prostatitis, and BPH by differentiating and quantifying cell size and morphology.

For example, FIGS. 38A-38C show mpMRI images obtained from a PCa patient. Patient information includes: age 63 years, PSA level 78.74 ng/mL, GS 3+4, and stage T2C. FIG. 38A shows a T2W image, FIG. 38B shows a DTI-ADC image, and FIG. 38C shows an H&E stained image of the prostate. The T2W and ADC images detected pathologist-identified PCa and regions of unidentified hypo-intense lesions (arrows 3800 in FIGS. 38A and 38B). Other factors may also affect T2W and ADC signal appearance.

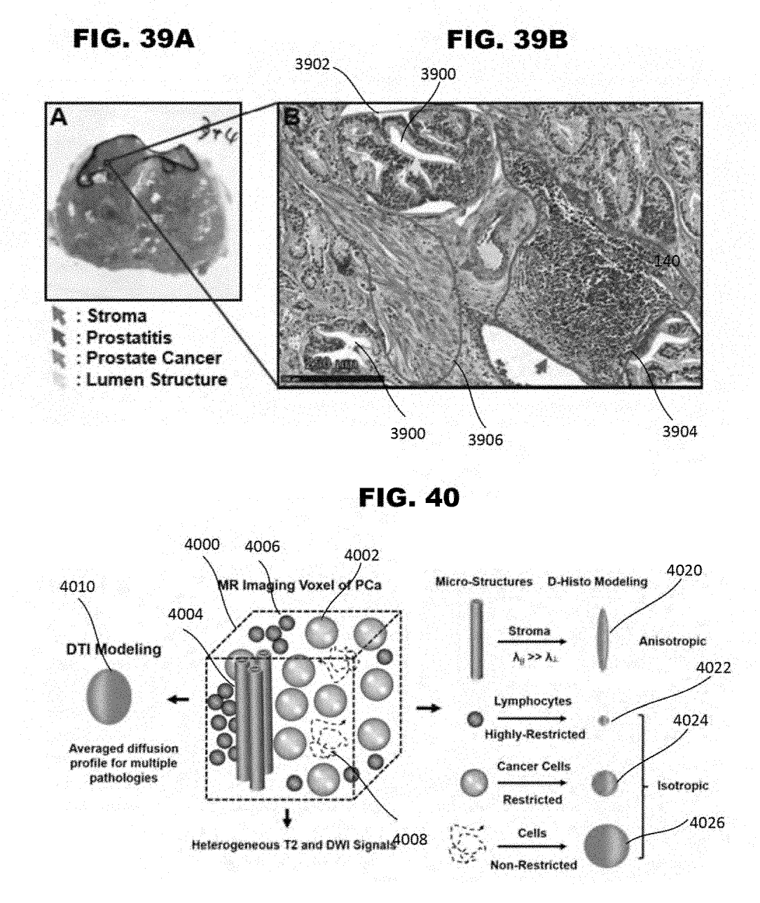

Within the prostate, multiple coexisting pathologies and structures may be present when making a PCa diagnosis (e.g., stromal BPH, prostatitis, and/or lumen water in addition to PCa cells). When using mpMRI alone to diagnose the prostate, these pathologies and structures may be indistinguishable, and may result in false-positive diagnoses for PCa. The current ‘clinical gold standard’ is a marked region (or regions) on an H&E stained microscope slide as determined by a trained pathologist. FIG. 39A is microscope image with a pathologist-marked region of PCa. FIG. 39B is an expanded portion of the marked region in FIG. 39A which constitutes less than a 5% in-plane area of an MRI voxel, obtained using the histology. While the pathologist-marked region is currently the “gold standard” diagnosis of the presence of PCa, the expanded pathologist identified cancer portion shows the coexistence of PCa, inflammation (prostatitis) and general stroma.

FIG. 40 is a schematic illustration of D-Histo model components in an imaged voxel 4000. Within the imaged voxel of tissue 4000 thought to contain prostate cancer, microstructures in addition to PCa cells 4002 may be present including, but not limited to, stroma 4004, inflammatory cells (lymphocytes) 4006, and lumen water 4008. The imaged voxel 4000 is produced by data from the DBSI method from MRI data according to:

S_{K}=fe^{-|\vec{b}}^{_{K}}^{|·(λ}^{_{∥}}^{-λ}^{_{⊥}}^{)cos} ^{²}^{_{ψK}}+∫ₐ^{b}f(D)e^{-|\vec{b}}^{_{K}}^{|D}dD(K=1,2,3, . . . ) (Equation 5)

In Equation 5, S_{K} is the measured diffusion weighted signal at diffusion gradient direction \vec{b}_{K} k; f is the signal fraction of the \vec{b}_{K} anisotropic diffusion component; \vec{|b_{K}|} is the diffusion weighting factor, ψ is the angle between the fiber and the diffusion gradient \vec{|b_{K}|} direction; and D is the apparent diffusion coefficient of isotropic diffusion components. The DWI/DTI method produces an averaged diffusion profile 4010 for multiple pathologies, which does not result in images of other microstructures. The D-Histo technique produces an anisotropic diffusion profile 4020 for stroma, an isotropic diffusion profile 4022 for inflammatory cells, an isotropic diffusion profile 4024 for prostate cancer cells, and an isotropic diffusion profile 4026 for lumen.

While other imaging methods such as DTI may generalize a diagnosis to PCa, D-Histo modeling can be used to differentiate between the microstructures present in the voxel by identifying and distinguishing an anisotropic diffusion tensor (representative of stroma), a highly restricted isotropic diffusion tensor (representative of inflammatory cells), a restricted isotropic diffusion tensor (representative of PCa cells), and a non-restricted isotropic diffusion tensor (including lumen water and normal prostate tissues). The relative abundance of each of these parameters may be expressed as a fractional contribution of each component to the overall diffusion MRI signal used to perform DBSI as described herein above, and by extension D-Histo as disclosed herein.

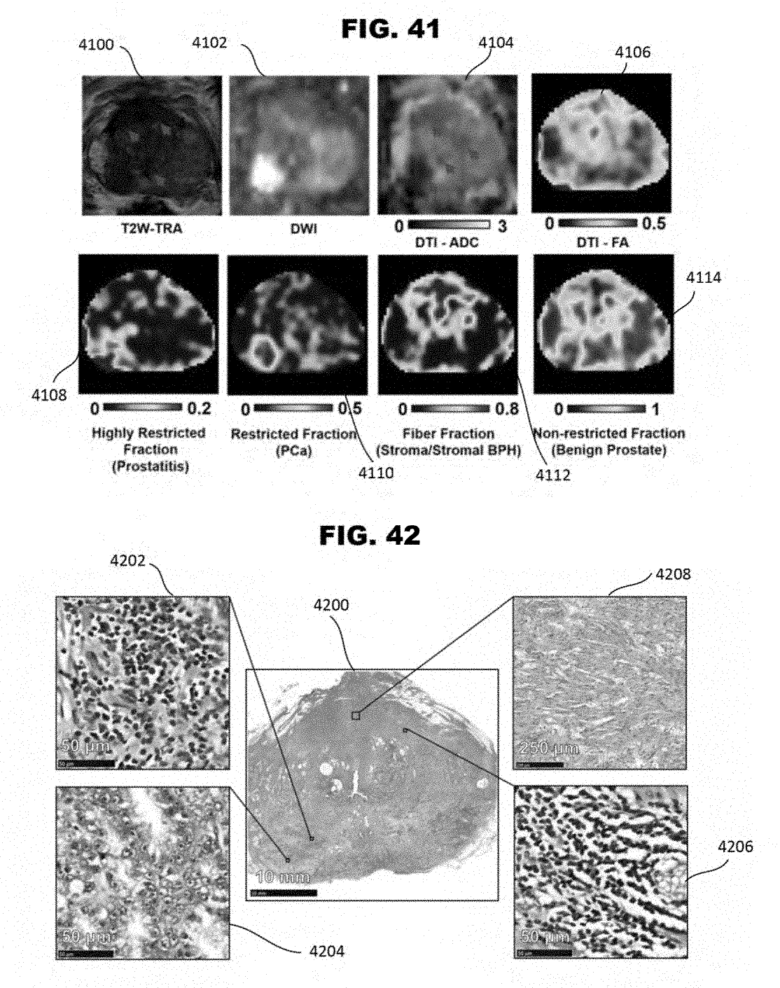

FIG. 41 is series of images comparing mpMRI and D-Histo results for PCa detection by way of non-limiting example. In this example, patient information includes: age 58 years, PSA level 16.49 ng/mL, GS 3+4, and stage T3A. Multi-parametric MRI prostate images 4100, 4102, 4104 and 4106 are shown on the top row, and D-Histo prostate images 4110, 4112, 4114 and 4116 are shown on the bottom row. The MRI prostate image 4100 is produced using T2W imaging. The MRI prostate image 4102 is produced using diffusion-weighted imaging (DWI). The MRI prostate image 4104 is produced by using an apparent diffusion coefficient in diffusion tensor imaging. The MRI prostate image 4106 is produced by using a diffusion tensor imaging using fractional anisotropy (FA). The D-Histo generated image 4110 is generated with a highly restricted isotropic diffusion tensor (representative of inflammatory cells). The D-Histo generated image 4112 is generated with a restricted isotropic diffusion tensor (representative of PCa cells). The D-Histo generated image 4114 is generated with an anisotropic diffusion tensor (representative of stroma). The D-Histo generated image 4116 is generated with a non-restricted isotropic diffusion tensor (representative of lumen water and normal prostate tissues). The hypo-intense T2W imaging and hyper-intense diffusion-weighted imaging (DWI) identified PCa region images 4100 and 4102 is consistent in location and size. In contrast, the D-Histo method reveals that in this mpMRI identified PCa region both inflammation (image 4110) and PCa (image 4112) coexist, consistent with the findings seen in haemotoxylin and eosin staining shown in FIG. 39B. D-Histo reveals the presence of stroma/stroma BPH (image 4114) in the “unidentified dark regions” remote to mpMRI identified PCa, arrows in the T2W imaging and DWI images 4100 and 4102.

FIG. 42 is a series of microscopic images of a whole mount section haemotoxylin and eosin stain as obtained from a patient with Gleason Score 3+4 from whom the images in FIG. 41 were obtained. An haemotoxylin and eosin stain image 4200 is shown at 0.3× resolution with magnified area images 4202 at 40×, 4204 at 40×, 4206 at 40× and 4208 at 8×. The haemotoxylin and eosin staining supports the results obtained using D-Histo technique described herein, including discovery of underlying chronic prostatitis (upper left and lower right images 4202 and 4204), PCa (lower left image 4206), and stromal BPH (upper right image 4208).

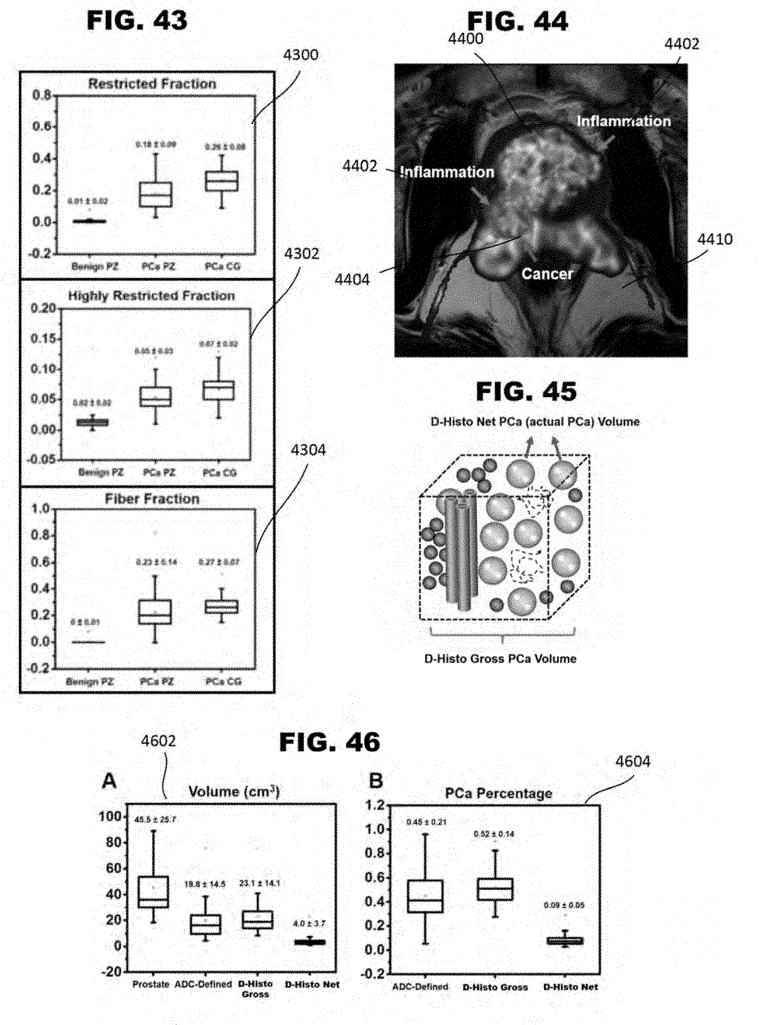

In this example, an experienced radiologist identified PCa regions from 49 subjects based on mpMRI findings. FIG. 43 shows graphs 4302, 4304 and 4306 that demonstrate the false positive of using mpMRI for identifying prostate cancer cells in the 49 patients. The identified cancer cells using mpMRI actually result from PCa, stroma/stromal BPH, and inflammation, in three mpMRI defined tissue types: benign peripheral zone tissue, peripheral zone prostate cancer tissue, and central gland prostate cancer tissue. The mpMRI detected PCa in peripheral zone (PZ) and central gland (CG) areas actually contained D-Histo-classified PCa (restricted diffusion), inflammation (highly restricted diffusion), and stroma (anisotropic diffusion). In contrast, the radiologist using mpMRI identified a benign prostate was free from these complications. These D-Histo results demonstrated the underpinning of false-positive PCa diagnosis using mpMRI. Peripheral zone PCa included 81 foci, central gland PCa included 91 foci, and benign peripheral zone tissue regions included 31 foci.

In various aspects, D-Histo data may be used to enable prostate disorder diagnosis and treatment management including, but not limited to screening, guiding biopsy, and focal therapy. Following a confirmation of positive PSA level, a precise imaging guided biopsy can be performed. Navigation can also be provided for radiation therapy (e.g., intensity-modulated radiation therapy and proton therapy), brachytherapy, cryogen therapy, and HIFU (high intensity focused ultrasound) therapy. Treatment evaluations and follow-up include guidance for active surveillance over the treatment process including the D-Histo technique are described herein. FIG. 44 is a 3-D reconstruction of a prostate 4400 with labelled inflammation regions 4402 and PCa regions 4404, overlaid on an anatomic T2-weighted image 4410 of surrounding structures that may be used to guide biopsy or radiation therapy by way of non-limiting example.

FIG. 45 is an illustration of components of a voxel volume. The mpMRI simply defines PCa volume as the sum of all PCa-containing (defined by ADC threshold) voxel volume. In contrast, D-Histo-Gross measurements define PCa volume as the sum of voxel volumes from image voxels containing>5% of a restricted fraction. D-Histo-Net measurements define PCa volume as the voxel volume multiplied by the sum of the restricted fractions. For all calculations, regardless of imaging method, the prostate volume is defined as the sum of voxel volume of the entire prostate. Thus, the D-Histo technique allows a more accurate determination of the actual PCa cells in contrast to other microstructures in the voxel.

FIG. 46 shows a graph of PCa volume 4602 and a graph of percentage results 4604 both determined using mpMRI as described herein above (labelled as “ADC-defined”) with respect to D-Histo measured PCa volume and percentage (labeled as “D-Histo Gross” and “D-Histo Net”). In one aspect, gross PCa volume calculations enable longitudinal assessment of an individual subject for PCa progression or treatment efficacy. In another aspect, PCa Percentage is defined as the gross PCa volume divided by the whole prostate volume, thus allowing for inter-subject comparison.

Without being limited to any particular theory, gross PCa volume measures the actual tumor distribution and extension within the prostate, and may be used to assess how widespread a PCa has invaded the prostate, as well as to stage the PCa. Net PCa volume measures total tumor cell volumes within the prostate of the patient. In an aspect, the sub-voxel resolution of the D-Histo method enables a non-invasive means of assessing total tumor cell volumes for an in vivo human prostate, whereas previously total tumor cell volumes were assessed using traditional mpMRI measurements of prostate. In one aspect, the net PCa volume may be a biomarker that reflects the severity of prostate cancer since partial volume effects of other imaging methods are eliminated by using D-Histo to greatly reduce the incidence of false positive signals associated with BPH and prostatitis conditions.

In some aspects, the disclosed D-Histo protocol generated DWI data may replace the DWI component of mpMRI from the PCa patients. Data from 172 de-identified imaging datasets of patients (including 50 prostatectomy patients, aged 57-89 years) with prostate diseases have been used in exemplary embodiments described herein. Diffusion-weighted MRI data was obtained on a 3T Siemens Skyra scanner (Erlangen, Germany) with an 18-channel phased-array body receive coil. The imaging parameters included: TR 5000 ms, TE 88 ms, 4 averages, FOV 112×140 mm², in-plane resolution 2×2 mm², 24 slices at 4-mm thickness, 25-dir icosahedral diffusion encoding scheme with maximum b-value 1500 s/mm.

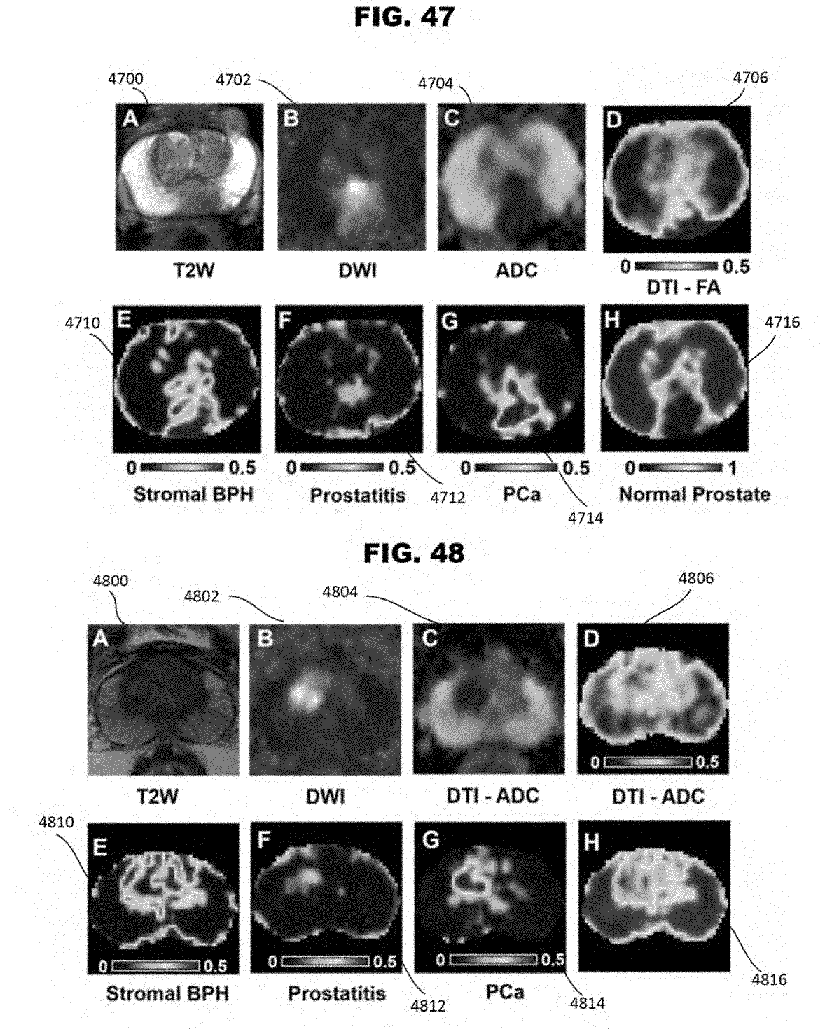

FIG. 47 is series of images comparing mpMRI (top row) and D-Histo results (bottom row) for PCa detection by way of non-limiting example. In this example, the prostate contained transition zone PCa, as obtained from a 67-year old patient who had undergone a standard 12-needle TRUS-guided biopsy. Multi-parametric MRI prostate images 4700, 4702, 4704 and 4706 are shown on the top row, and D-Histo prostate images 4710, 4712, 4714 and 4716 are shown on the bottom row. The MRI prostate image 4700 is produced using T2W imaging. The MRI prostate image 4702 is produced using diffusion-weighted imaging (DWI). The MRI prostate image 4704 is produced by using an apparent diffusion coefficient derived by diffusion tensor imaging. The MRI prostate image 4706 is produced by using a diffusion tensor imaging derived fractional anisotropy (FA). The D-Histo generated image 4710 is generated with an anisotropic diffusion tensor fraction map (representative of stroma). The D-Histo generated image 4712 is generated with a highly restricted isotropic diffusion tensor fraction map (representative of inflammatory cells). The D-Histo generated image 4714 is generated with a restricted isotropic diffusion tensor fraction map (representative of PCa cells). The D-Histo generated image 4716 is generated with a non-restricted isotropic diffusion tensor fraction map (including lumen water and normal prostate tissues).

Referring to FIG. 47, the prostatectomy patient had significant lesions in the prostate showing positive PCa at cores from needles 1, 6, 7, 8, 12 with Gleason scores of 3+3 on all 5 cores. Suspected PCa based on DWI and ADC, derived by mpMRI, is visible in the transition zone (images 4702 and 4704). The bottom row of images 4710, 4712, 4714 and 4716 include maps of D-Histo metrics for the patient derived from the DWI data using D-Histo analysis techniques. The putative inflammation as represented by highly restricted isotropic diffusion (image 4712, 0≦ADC<0.1) and PCa represented by restricted isotropic diffusion (image 4714, 0.1<ADC<0.7) are quantified by the voxel-wise isotropic diffusion spectrum, wherein the proportion of small cells and big cells within each voxel may be distinguished by sub-ranges of ADC within the isotropic diffusion spectrum. The putative BPH in mpMRI was quantified as high anisotropy (FA) on a DTI map (image 4706), and was quantified using the D-Histo anisotropic diffusion tensor fraction (image 4714), which reflects the more fiber-like nature of stromal tissues as compared to normal prostate, PCa, and lymphocytes. The image of putative normal prostate (image 4716, ADC>0.7) includes regions of prostate that have normal ADC values.

FIG. 48 is series of images comparing mpMRI (top row) and D-Histo results (bottom row) for PCa detection by way of non-limiting example. In this example, the prostate included peripheral and transition zone PCa, as obtained from a 69-year old prostatectomy patient who also underwent the standard 12-needle biopsy (needles 3, 7, 8, 9 positive with Gleason=4+4 on all core samples). Multi-parametric MRI prostate images 4800, 4802, 4804 and 4806 are shown on the top row, and D-Histo prostate images 4810, 4812, 4814 and 4816 are shown on the bottom row. The MRI prostate image 4800 is produced using T2W imaging. The MRI prostate image 4802 is produced using diffusion-weighted imaging (DWI). The MRI prostate image 4804 is produced by using an apparent diffusion coefficient in diffusion tensor imaging. The MRI prostate image 4806 is produced by using a diffusion tensor imaging using FA. The D-Histo image 4810 is generated with an anisotropic diffusion tensor fraction map (representative of stroma). The D-Histo image 4812 is generated with a highly restricted isotropic diffusion tensor fraction map (representative of inflammatory cells). The D-Histo image 4814 is generated with a restricted isotropic diffusion tensor fraction map (representative of PCa cells). The D-Histo image 4816 is generated with a non-restricted isotropic diffusion tensor fraction map (including lumen water and normal prostate tissues). Suspected PCa based on D-Histo was detected at the peripheral and transition zones as shown in image 4814.

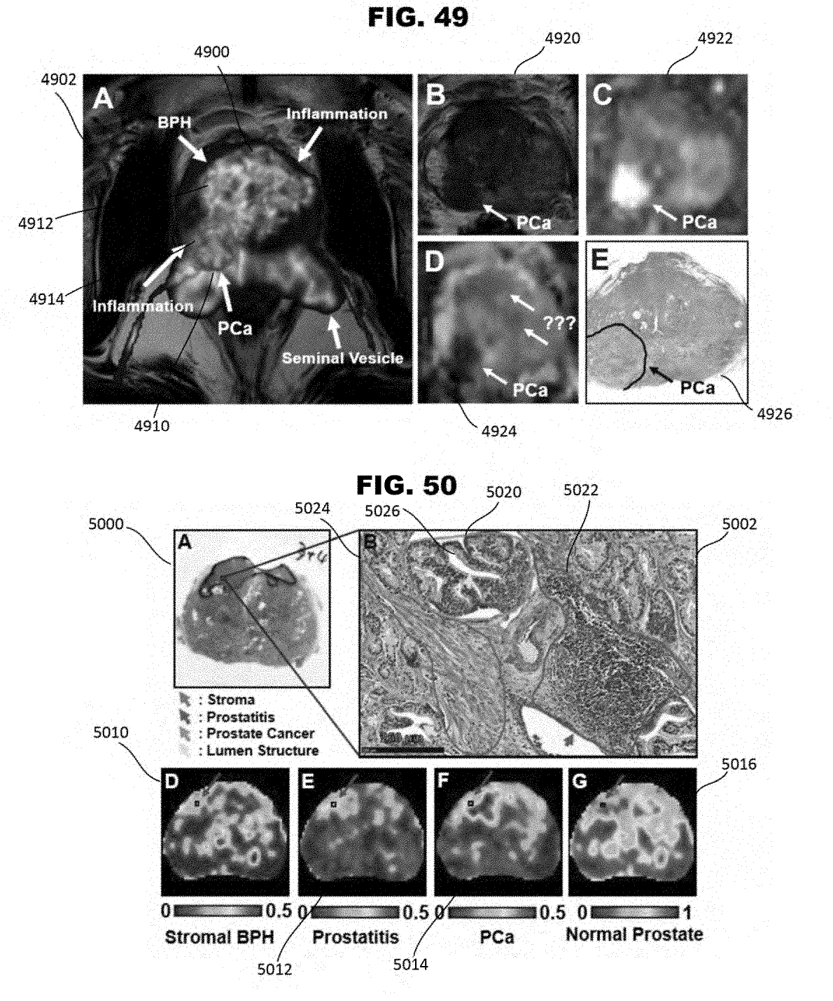

FIG. 49 is a 3D-rendered prostate image 4900 overlaid on an anatomical image T2W 4902, as obtained from a 58-year old patient who underwent prostatectomy and needle biopsy (Gleason scorer 3+4). Suspected PCa cells 4910 located to the peripheral zone (lower right corner; pink) with transition zone BPH 4912 (gold) and peripheral zone inflammation 4914 (blue-green) as shown in FIG. 49. In contrast, images 4920, 4922, and 4924 are generated from mpMRI and image 4926 is H&E staining. Some embodiments of the methods and systems described herein include 3D rendering of the prostate to match the theoretical TRUS-guided needle biopsy locations to correlate Gleason scores of individual biopsy cores with D-Histo metrics.

FIG. 50 shows an image 5000 of a whole mount H & E slide and a magnified image 5002 with D-Histo maps 5010, 5012, 5014 and 5016. The D-Histo generated image 5010 is generated with an anisotropic diffusion tensor fraction map (representative of stroma). The D-Histo generated image 5012 is generated with a highly restricted isotropic diffusion tensor fraction map (representative of inflammatory cells). The D-Histo generated image 5014 is generated with a restricted isotropic diffusion tensor fraction map (representative of PCa cells). The D-Histo generated image 5016 is generated with a non-restricted isotropic diffusion tensor fraction map (including lumen water and normal prostate tissues). FIG. 50 shows in vivo D-Histo metric maps 5010, 5012, 5014 and 5016 of a prostate compared with a corresponding histology for PCa in the transition zone of prostate identified by mpMRT and whole mount pathology. By examination of the diffusion MRI results using D-Histo to target cell size and morphology difference among PCa, inflammation, and BPH, overlapping regions of PCa 5010, inflammation 5012, BPH 5014 and lumens 5016 were identified in a transition zone shown in magnified image 5002 that was missed by the mpMRT technique.

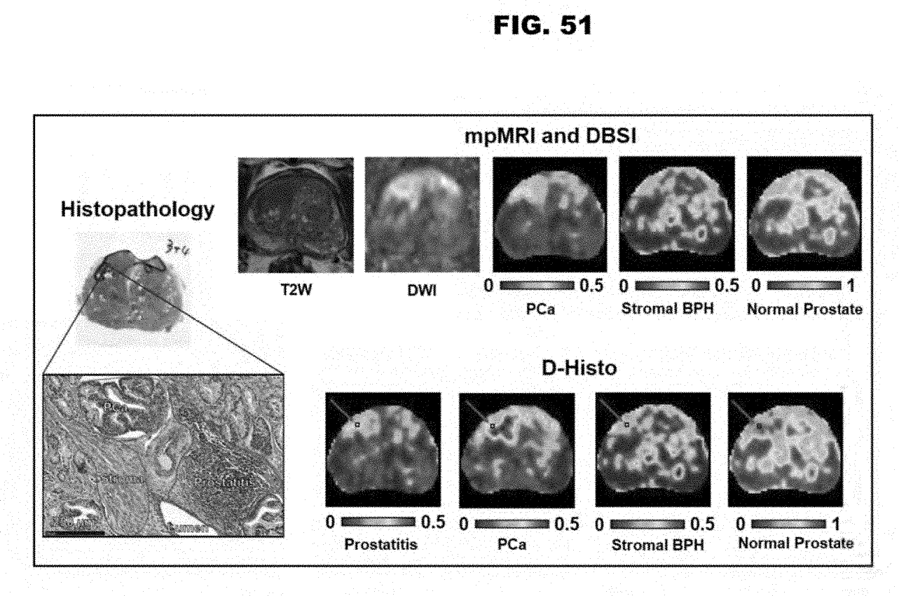

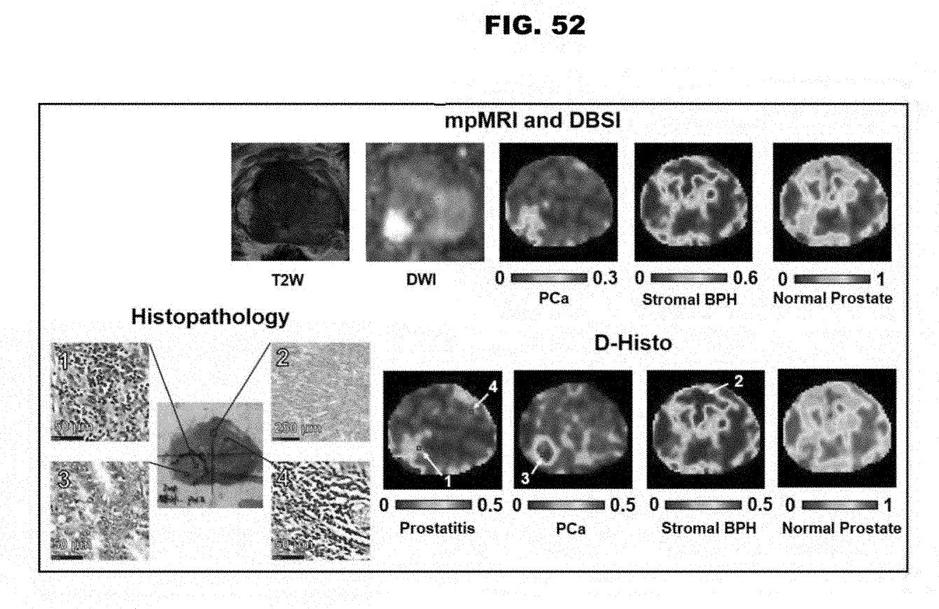

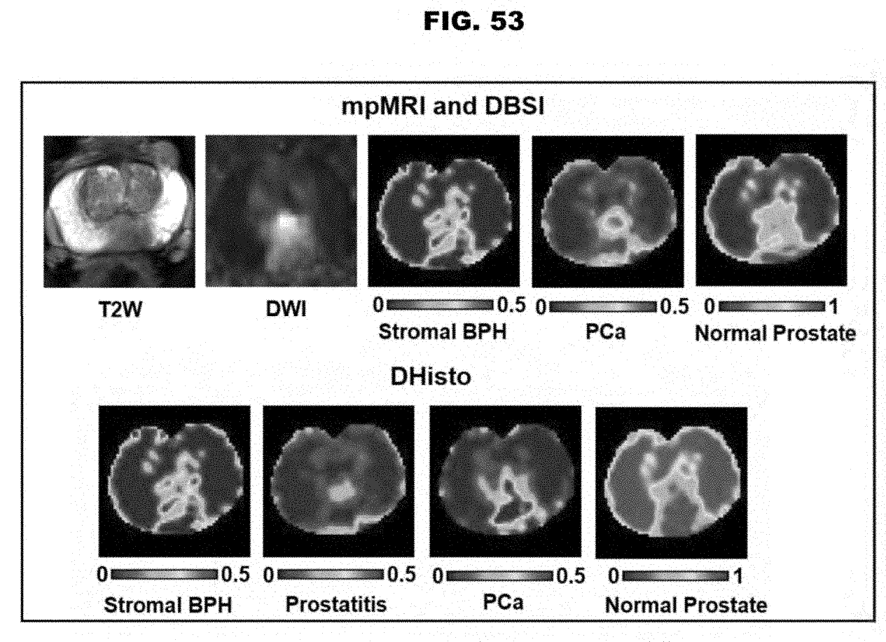

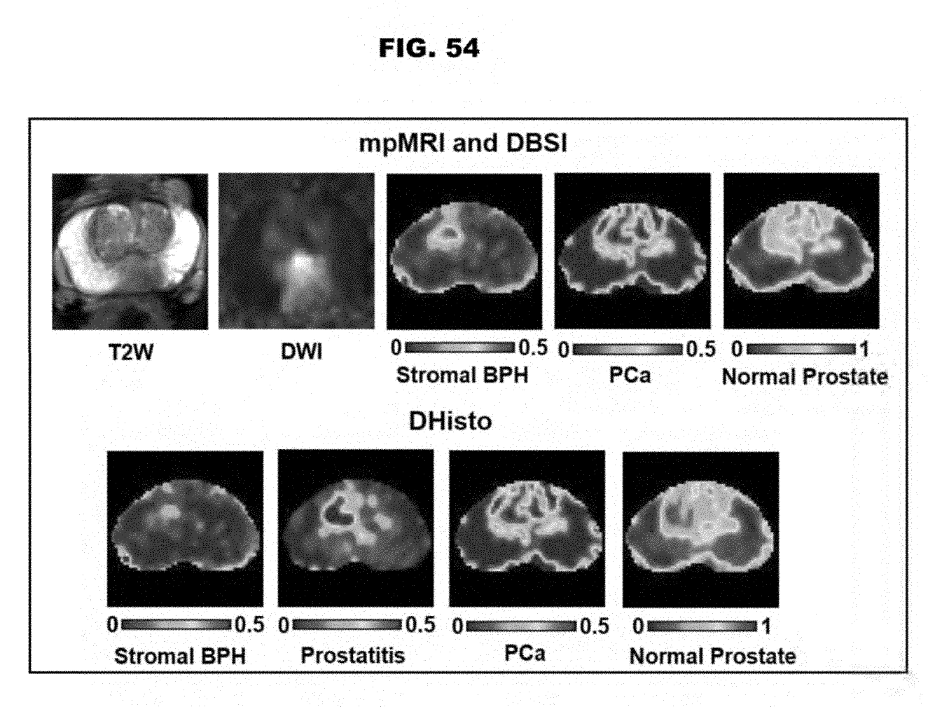

FIGS. 51-54 are additional series of images comparing mpMRI, DBSI, and D-Histo pathological metrics measured from the prostates of four additional patients. FIGS. 51-53 each show images of a whole mount H & E slide and a magnified image of prostates. In each of these examples shown in FIGS. 51-54, PCa identified by mpMRI (images using T2W and DWI in the top row) and DBSI (images in top row) was compared with the more refined classification enabled by D-Histo (bottom row images produced using highly restricted isotropic diffusion tensors, restricted isotropic diffusion tensor, anisotropic diffusion tensor and non-restricted isotropic diffusion tensor) to demonstrate the previously established DBSI for CNS pathological analysis fails to detect inflammation and a portion of PCa thus both over- and under-estimating the extent of PCa. A combination of raw DBSI output, simulation results, and pathological staining allows for identification of lymphocytes, as well as separation of lymphocytes from PCa using D-Histo classification.

In summary, D-Histo biomarkers as described herein have robustly diagnosed PCa, as differentiated from other prostate disorders including prostatitis and BPH. Non-limiting examples of the D-Histo biomarkers include (but not limited to): restricted isotropic diffusion fraction derived from the isotropic diffusion spectrum representing PCa cell fraction (0.1≦ADC≦0.7), highly restricted isotropic diffusion fraction derived from the isotropic diffusion spectrum representing inflammatory cell fraction (0<ADC<0.1), non-restricted isotropic diffusion fraction derived from the isotropic diffusion spectrum representing normal prostate lumen water fraction (0.7<ADC<3.5), and fiber fraction derived from the anisotropic diffusion tensors representing stroma fraction. In various aspects, the non-restricted, restricted, and highly restricted isotropic diffusion fractions are identified within the isotropic diffusion spectrum using predefined threshold parameters identifying a subrange of the isotropic diffusion spectrum. These threshold values may be provided using any known means including, but not limited to, estimation from cellular dimensions and structures, published data such as animal studies, analysis of DBSI/D-Histo measurements of calibration samples with a known distribution of known cells, in silico simulation, and any other suitable means. Accurate identification and quantification of prostate disorders is advantageous for avoiding false positive diagnoses, as well as for application of more efficient treatment strategies.

D-Histo may noninvasively assess Gleason grades of PCa using machine learning (e.g., via a Support Vector Machine (SVM)), analysis on D-Histo derived multiple metrics. Patients with high PSA level commonly undergo 12-core biopsy to confirm the presence of PCa. In a cohort of 62 patients undergoing D-Histo and mpMRI examinations, the biopsy needle locations and pathological Gleason score were also registered. Taking the approximate needle locations and assessed Gleason scores, D-Histo metric features were analyzed using SVM. Results revealed that D-Histo with SVM achieved high accuracy (86.6% vs. 14.5% for ADC by mpMRI) in matching all biopsy Gleason grades with the following individual accuracy: Gleason score≦6 (90.4% vs. 44.3% for ADC by mpMRI), 3+4=7 (87.8% vs. 36.6% for ADC by mpMRI), 4+3=7 (82.5% vs. 12.5% for ADC by mpMRI), 3+5 (92.5% vs. 0% for ADC by mpMRI), 4+5 (84.6% vs. 1% for ADC by mpMRI), 5+4 (90.9% vs. 54.2% for ADC by mpMRI), and 5+5 (97% vs. 39% for ADC by mpMRI). D-Histo with SVM also performed accurately in National Comprehensive Cancer Network (NCCN) prostate cancer risk stratification: Overall (91.5% vs. 43.8% for ADC by mpMRI), Low risk (87.5% vs. 44% for ADC by mpMRI), Intermediate risk (91.9% vs. 60.3% for ADC by mpMRI), High risk (91.1% vs. 3.5% for ADC by mpMRI), and Very High risk (91.8% vs. 6.3% for ADC by mpMRI). When looking at the contemporary PCa Grading System, the D-Histo method still outperforms mpMRI derived ADC as follows: Overall (85.5% vs. 44.1% for ADC by mpMRI), Grade Group 1 (89.4% vs. 44.0% for ADC by mpMRI), Grade Group 2 (90.8% vs. 35.7% for ADC by mpMRI), Grade Group 3 (84.9% vs. 19.3% for ADC by mpMRI), Grade Group 4 (87.5% vs. 11.1% for ADC by mpMRI), and Grade Group 5 (84.0% vs. 60.3% for ADC by mpMRI).

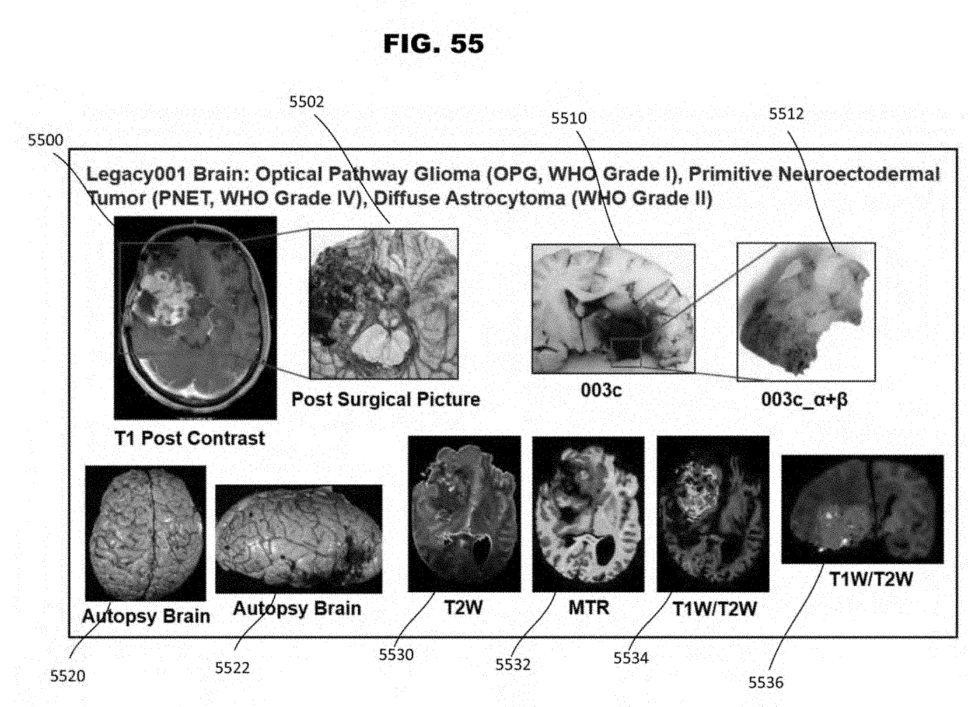

In another aspect, the D-Histo method described herein above for the diagnosis of prostate disorders may be modified to enable diagnosis of various brain tumors (glioblastoma). Previous methods, illustrated by various images shown in FIG. 55, may experience limitations with respect to differentiating brain tumor cells from surrounding cells in a manner similar to the differentiation of PCa cells from surrounding cells in the prostate. The images in FIG. 55 were taken from an autopsy brain specimen from a patient diagnosed with optical pathway glioma (OPG, WHO Grade I), primitive neuroectodermal tumor (PNET, WHO Grade IV) and diffuse Astrocytoma (WHO Grade II). FIG. 55 shows a T1 post contrast image 5500 with a postmortem fresh specimen 5502 of one region of the image 5500. FIG. 55 also shows a postmortem specimen after formalin fixation 5510 of a tissue slice of the brain and a tissue block 5512 from one of the regions of the fixed tissue specimen 5510. Images 5520 and 5522 are photographs of the autopsy brain. An image 5230 of the brain is generated using T2-weighted imaging, an image 5232 of the brain is generated using magnetization transfer ratio (MTR), and images 5234 and 5236 of the brain are generated using combined T1 and T2 weighted imaging.

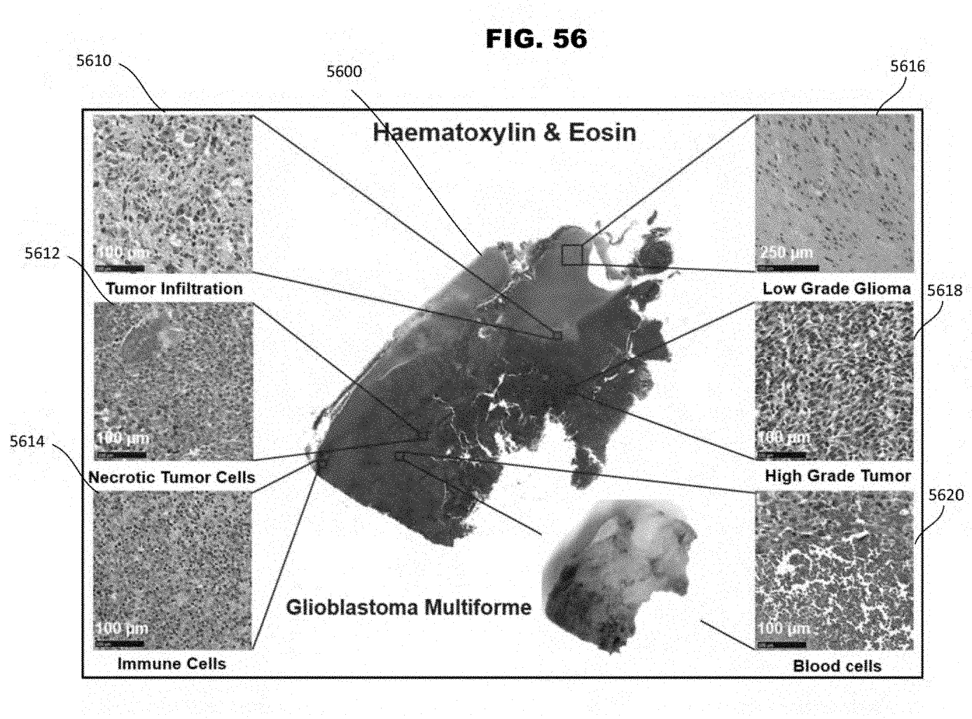

FIG. 56 shows a haemotoxylin and eosin (H&E) staining image 5600 of a slice of brain tissue at lx magnification. A closeup image 5610 of one region of the image 5600 at 20× magnification shows tumor infiltration. A closeup image 5612 of one region of the image 5600 at 20× magnification shows necrotic tumor cells. A closeup image 5614 of one region of the image 5600 at 20× magnification shows immune cells. A closeup image 5616 of one region of the image 5600 at 8× magnification shows low grade glioma. A closeup image 5618 of one region of the image 5600 at 20× magnification shows a high grade tumor. A closeup image 5620 of one region of the image 5600 at 20× magnification shows blood cells. As illustrated in FIG. 56, the cells within a region of the brain tumor may include inflammatory cells, red blood cells associated with hemorrhage, and white matter tracts (not shown). Referring again to FIG. 56, there may also be different arrangements of brain tumor cells that may provide information useful for staging or classifying the type of brain tumor present in the patient, including, but not limited to infiltrating tumor cells, necrotic tumor cells, low-grade gliomas, and glioblastomas.

In the DBSI analysis described herein above, two shortcomings limited the ability of DBSI to identify specific tissue structures: (1) using the fixed single isotropic ADC to identify infiltrating cancer and inflammatory cells, and (2) employing a fixed uniformly defined basis set to model the diffusion weighted MRI signals. The predefined thresholds described herein above to identify the non-restricted, restricted, and highly restricted isotropic diffusion fractions within the isotropic diffusion spectrum used to categorize prostate cells using D-Histo may be modified to enable the detection of brain tumor cells and other surrounding structures by adapting D-Histo/PCa analysis approach with an additionally data-driven basis set based on diffusion weighted MRI signal patterns.

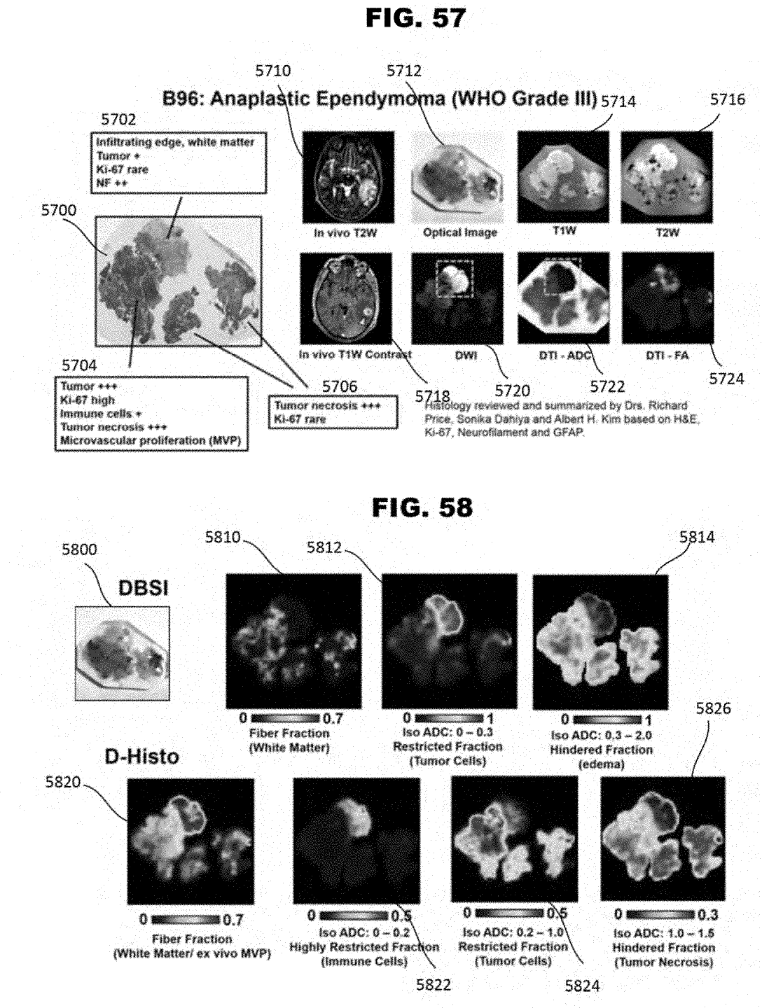

By way of non-limiting example, FIG. 57 shows images from a case of anaplastic ependymoma (WHO Grade III) showing the corresponding histology and mpMRI maps. Specifically, FIG. 57 shows a haemotoxylin and eosin image 5700 of brain tissue that shows a first region 5702 that includes an infiltrating edge, white matter and tumor cells. A second region 5704 includes a high concentration of tumor cells and tumor necrosis (necrotic tumor cells) as well as immune cells and microvascular proliferation. A third region 5706 includes a high concentration of tumor necrosis. FIG. 57 shows an in vivo image 5710 generated by T2-weighted imaging, a photo of specimen 5712, an image 5714 generated by T1-weighted imaging, an image 5716 generated by T2-weighted imaging, an in vivo image 5718 generated by T1-weighted imaging with contrast enhancement, an image 5720 generated by diffusion-weighted imaging, an image 5722 generated by diffusion tensor imaging derived apparent diffusion coefficient, and an image 5724 generated by diffusion tensor imaging derived FA.

FIG. 58 shows a photo 5800 of a brain tissue block. A DBSI image 5810 is generated with a fiber fraction representing white matter. A DBSI image 5812 is generated using a restricted fraction of an apparent diffusion coefficient between 0 and 0.3 representing tumor cells. A DBSI image 5814 is generated using a hindered fraction of an apparent diffusion coefficient between 0.3 and 2 representing edema. A D-Histo image 5820 is generated with a fiber fraction [representative of white matter or ex vivo microvascular proliferation (MVP)]. A D-Histo image 5822 is generated with a highly restricted fraction of 0 to 0.2 showing low grade glioma or immune cells. A D-Histo image 5824 is generated with a restricted fraction of 0.2 to 1 (representative of tumor cells). A D-Histo image 5826 is generated with hindered tensor fraction of 1.0 to 1.5 (representative of tumor necrosis). Comparing the results of the DBSI technique with those of the D-Histo technique on these specific histologically analyzed biopsy tissues as shown in FIG. 58, a stark contrast between the ability of the DBSI and D-Histo techniques to detect tissue structures within the brain was demonstrated. For example, the DBSI technique mistakenly identified white matter tracts as brain tumors while the D-Histo technique correctly reflects histology-identified tissue structures.

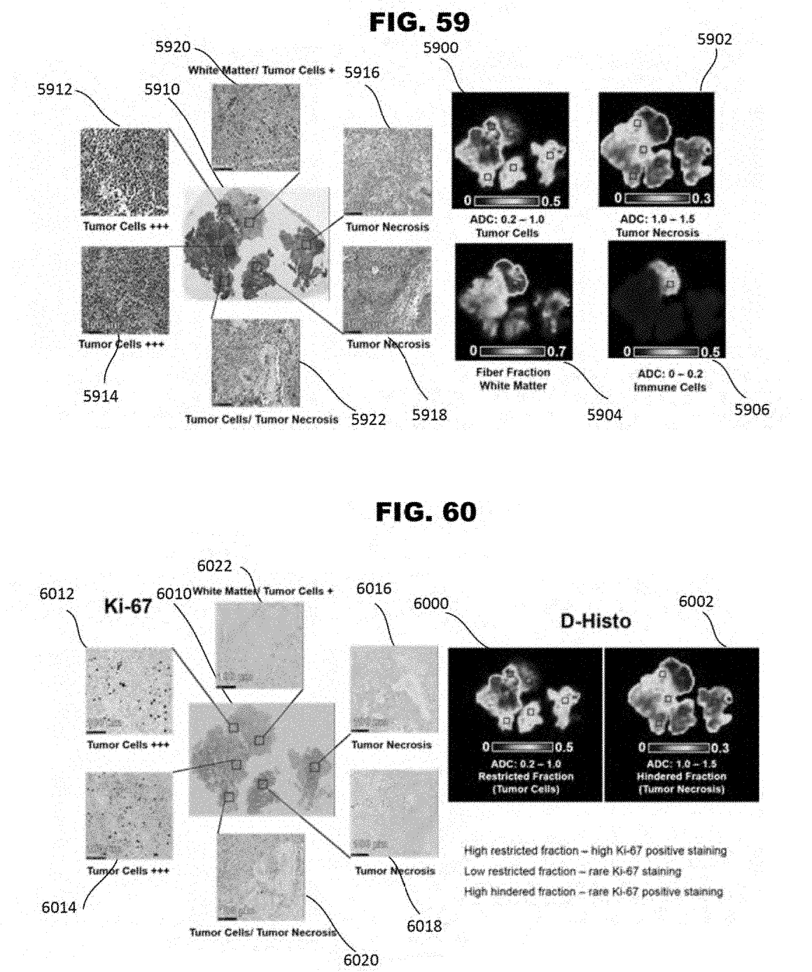

FIG. 59 shows a series of parameter maps 5900, 5902, 5904, and 5906, and an image 5910 of a corresponding histological slide stained using hematoxylin and eosin (H&E) showing several locations of brain tumor cells within the region of interest. Additional regions of the image 5910 are shown in magnified images 5912 and 5914 showing tumor cells, images 5916 and 5918 showing tumor necrosis, image 5920 showing white matter and tumor cells and image 5922 showing tumor cells and tumor necrosis. As illustrated in FIG. 59, the map of restricted isotropic diffusion fraction (0.2≦ADC≦1.0 μm²/ms) 5900 highlights regions of the brain containing tumor cells. Tumor necrosis (dead tumor cells only) can be identified by hindered isotropic diffusion fraction (1.0<ADC≦1.5 μm²/ms) in the map 5902, and necrotic tumor cells (both living and dead cells) may be identified by a slightly expanded hindered isotropic diffusion fraction (0.5<ADC≦1.5 μm²/ms). The narrower ADC range may be selected to artificially separate live from dead tumor cells in various aspects. Low-grade glioma cells and/or immune cells can be identified by highly restricted isotropic diffusion fraction (0<ADC≦0.2 μm²/ms) in the map 5906. Also demonstrated is the correctly identified white matter tracts characterized by a relatively high fiber fraction in the map 5904. This example demonstrates the limitation of diffusion MRI on definitively defining brain tumors and that additional parameters and analysis methods are needed.

FIG. 60 is a series of parameter maps 6000 and 6002 and a corresponding image 6010 from a histological slide stained using Ki-67 (a cellular marker for rapidly proliferating tumor cells) showing the location of brain tumor cells within the region of interest. Additional regions of the image 6000 are shown in magnified images 6012 and 6014 showing tumor cells, images 6016 and 6018 showing tumor necrosis, image 6020 showing white matter and tumor cells and image 6022 showing tumor cells and tumor necrosis. As illustrated in FIG. 60, the maps 6000 and 6002 of D-Histo-identified tumors highlights regions of the brain containing tumor cells and tumor necrosis respectively.

In various aspects, the D-Histo diagnostic method described herein is capable of imaging inflammation without the need to inject exogenous agents, and is further capable of distinguishing inflammation from cancers for cancer detection. In one aspect, the D-Histo techniques described herein, with appropriate modification, may be used to diagnose disorders characterized by histologically distinct regions within other tissues including, but not limited to, cardiac tissue. In this aspect, myocarditis (or inflammatory cardiomyopathy) based on the differences in structure of myocardial tissue (reflected by anisotropic diffusion with an ellipsoidal water displacement profile) and inflammation cells (reflected as restricted isotropic diffusion with a spherical water displacement profile) may be differentiated using modifications of the D-Histo method as disclosed herein. In another aspect, D-Histo may also be used to assess the extent of permanent damage of myocardium characterized by losing anisotropic diffusion fraction, as opposed to temporary dysfunction caused by inflammation characterized by restricted isotropic diffusion with a spherical water displacement profile, or myocardial remodeling after myocardial infarction (MI, i.e., heart attack), characterized by changes in anisotropic diffusion tensor magnitude and direction. Similar to PCa, these modified D-Histo tools can be used to detect, differentiate, and quantify inflammation for other types of cancers and solid tumors, including but not limited to brain, breast, cervical, and pancreatic cancers.

FIG. 61 compares conventional diffusion tensor imaging (DTI) derived fractional anisotropy (FA) and ADC maps 6100, 6102, 6104 and 6106 with D-Histo classified myocardial fiber density, inflammation, and edema maps 6110, 6112, 6114 and 6116 in a rat heart of myocardial infarction. The maps produced by the D-Histo technique clearly identified regions of permanent damage (loss of myocardial fiber density with increased edema extent) in contrast to potentially reversible injury (increased inflammation without loss of myocardial fiber density).

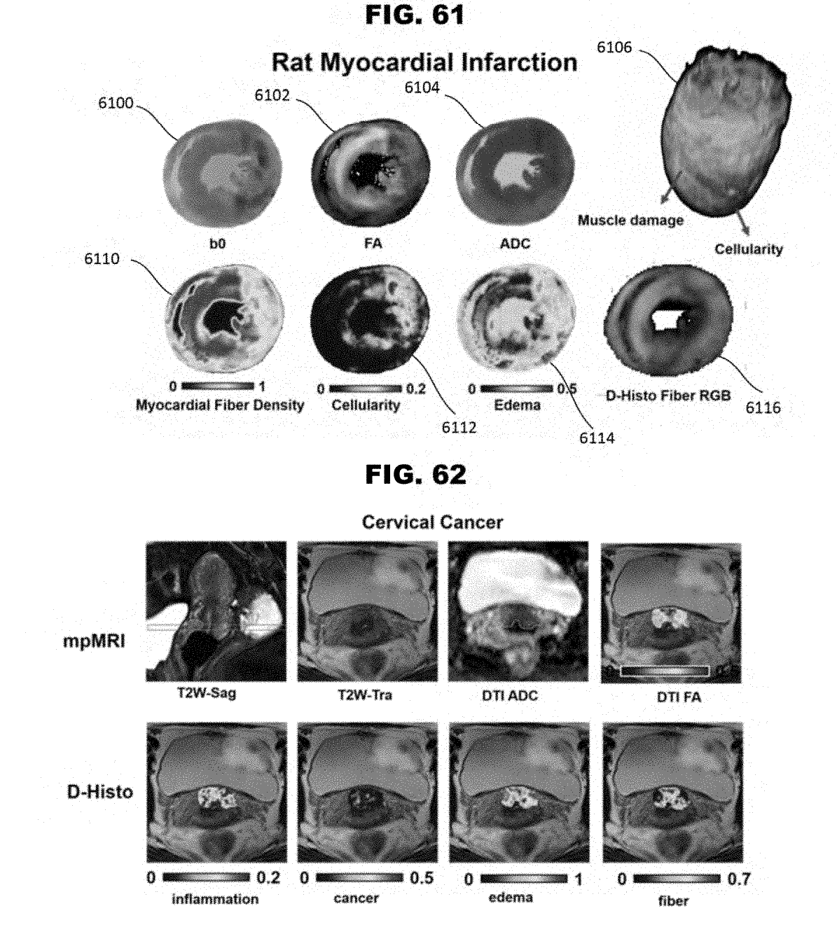

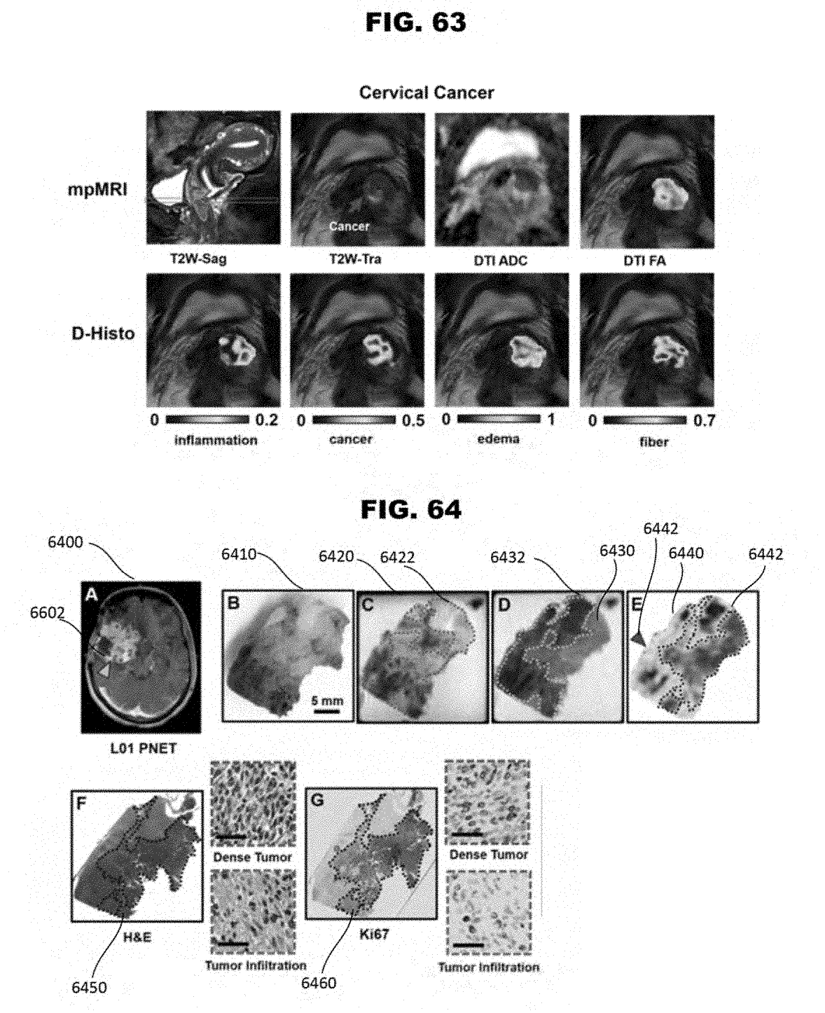

FIGS. 62-63 demonstrate the application of D-Histo in detecting cervical cancer in two different patients. Similar to prostate tissue classification, cervical tissues can be classified using the criteria previously established for PCa cases. Both FIGS. 62 and 63 show the classification of microstructures as inflammation, cervical cancer cells, edema or fiber. FIG. 63 shows concurrence in the detection of PCa between the D-Histo and mpMRI techniques. In FIG. 62, cervical cancer, erroneously detected by the mpMRI technique using a low ADC was correctly identified as inflammation by D-Histo.

FIG. 64 shows a series of MR images and histological staining for a pediatric primitive neuroectodermal tumor (PNET). An in vivo Gd-enhanced T1W image 6400 depicts heterogeneous contrast enhancements (arrowhead 6402). FIG. 64 shows an optical image 6410 of the gross appearance of autopsy brain specimen. An ex vivo T2W image 6420 reveals possible tumor distribution suggested by the widely accepted criterion of a significant decrease in ADC (dotted line region 6422). An ADC map 6430 suggests suspicious hyper cellularity regions (dotted line region 6432). A D-Histo derived tumor map 6440 suggests a completely different tumor distribution (dotted line region 6442) compared with the T2W image 6420 and the ADC map 6430. The D-Histo technique also successfully detected tumor infiltration to white matter tract shown by an arrow head 6444. H & E and Ki 67 staining images 6450 and 6460 show close tumor distribution with the D-Histo derived tumor map 6440.

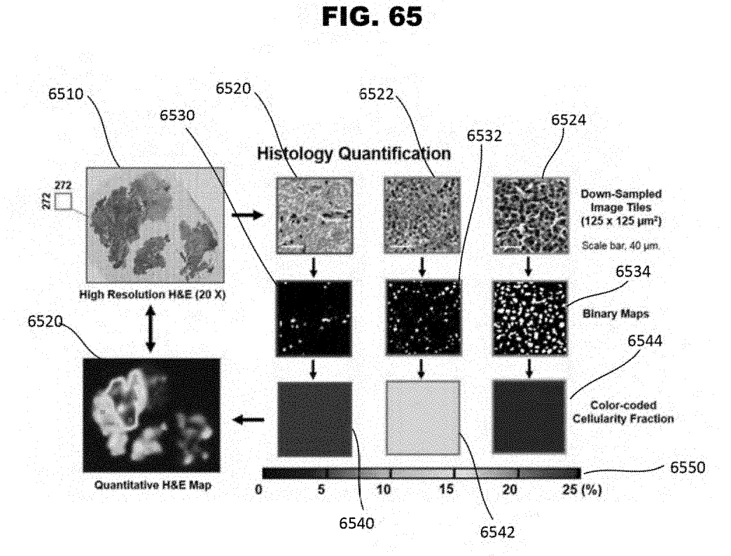

FIG. 65 is a series of distributions showing the process to produce a quantitative histology map 6500 from a high resolution H & E staining image 6510 of a tissue sample for comparison to the results of the D-Histo. In this process, the H & E stained image 6510 is divided into different imaging tiles such as the tiles 6520, 6522, and 6524. Each of the tiles in this example are 125×125 μm² in area in this example, but other resolutions may be selected. The individual tiles from the downsampled histology images 6520, 6522, and 6524 are projected back to the original high-resolution image 6500 and quantified by counting areas of positive staining with resulting masks. Binary maps such as maps 6530, 6532 and 6534 are created from each of the tiles 6520, 6522, and 6524 using the masks. The data in the binary masks is calculated and converted to a color-coded cellularity map. For example, the binary map 6530 is converted into a blue color tile 6540, the binary map 6532 is converted to a green color tile 6542 and the binary map 6534 is converted to a red color tile 6544. The color scale and corresponding percentage of cancer cells is shown in a scale 6550 in this example. Other microstructures may be substituted for cancer cells in a different scale to provide other histology maps. The color tiles such as the tiles 6540, 6542 and 6544 are then stitched back to form the downsampled quantitative histology map 6500. The process of automatically coding each tile or voxel improves accuracy over pathological analysis currently conducted by pathologists.

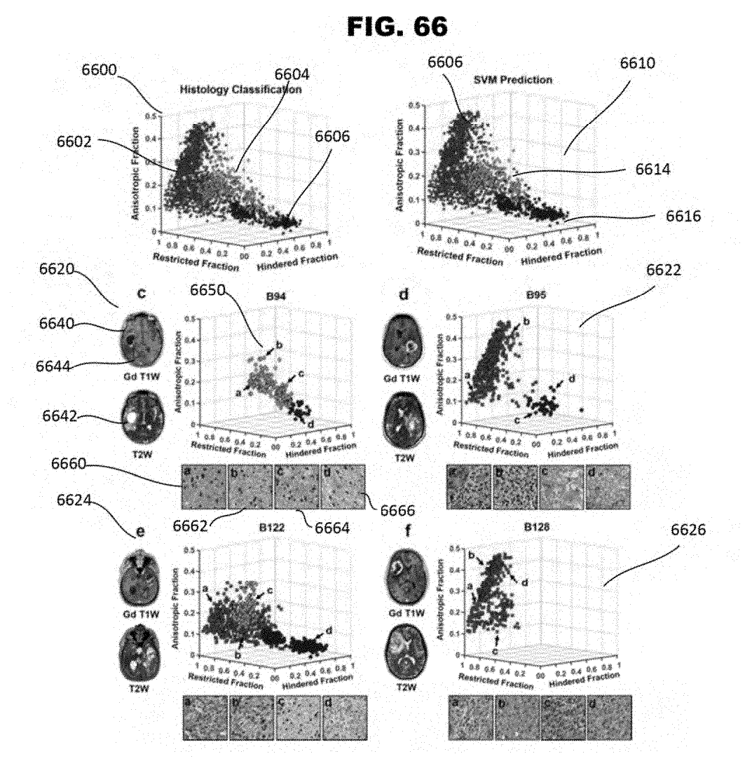

FIG. 66 shows the results of support vector machine (SVM) classification for dense tumor, tumor necrosis and tumor infiltration based on being taught from a training set of data derived from a histology classification. A classification plot 6600 shows histology classification for 1963 testing imaging voxels from 22 specimens of 14 different patients and 6975 image voxels based on the D-Histo technique. Each of the voxels is classified based on anisotropic fraction, restricted fraction and hindered fraction derived by the D-Histo technique in the plot 6600. The plot categorizes dense tumors (red squares 6602), necrosis (blue spheres 6604) and tumor infiltration (green diamonds 6606).

A supervised machine learning system, SVM, was taught on a set of categorized 5,000 voxels, equally divided into training and validation sets, from 18 specimens. A total of 7319 running combinations was employed to avoid training-prediction group selection bias. After the SVM completed learning on the training set, the classifiers were employed to classify D-Histo image data. An example plot 6610 shows a SVM classification of the same testing imaging voxels that produced the plot 6600. The plots 6600 and 6610 show high agreement and overall SVM classification accuracy was 95.6%. Dense tumor (red squares 6612), necrosis (blue spheres 6614) and tumor infiltration (green diamonds 6616) were classified by SVM with accuracies of 97.3%, 100% and 85.0%, respectively.

The learning process for the SVM inputs a set of training data made up of microstructure examples that are a result of the application of the D-Histo technique. The number of examples in the training data set depends on the number of weights and the desired accuracy (minimization of error). In this example, the learning process goes through a forward phase where weighting of links in a neural network is fixed. The nodes of the network are established in response to the modeling phase. The initial weighting factors are determined by the activation plotting function of each input node within a neural network. The neural network process is then implemented by propagating the input data from the training set through the network of nodes layer by layer to produce outputs. The neural network adjusts internally derived calculated weights between each of the established node connections by minimizing an error function against actual values during the training process. The output of the neural network is a predicted set of target values and associated weight ranges.

The forward phase finishes with the computation of an error value between the actual resulting outputs and the desired resulting outputs from the training set. The computation of the error value is the root mean square of the error calculated in the model prediction compared with the actual values. The SVM is then checked to determine whether the calculated outputs are sufficiently close to the actual outputs of the training set. If the SVM is sufficiently accurate as determined by a data scientist, the process ends. If the SVM is not sufficiently accurate, the process implements a backward phase where the error value is propagated through the network of nodes. Adjustments are made to the weighting factors to minimize the error between the actual output and the desired output in a statistical sense. The adjustments and the particular metrics for adjustment are made by a data scientist. The process then loops to the forward phase for further test data for refining the learning process. The weighting for the links of the network may also be adjusted by additional data to further refine the weighting values. Thus, the learning process may be run periodically with new data received to further teach the SVM.

FIG. 66 also shows plots based on SVM classification from image from a removed tumor and confirmation of classification based on enhanced images of different areas in the tumor for four patients 6620, 6622, 6624 and 6626. For example, the data from patient 6620 includes a Gd T1W based image 6640 and a T2W based image 6640 of a removed tumor. One area of the removed tumor 6644 was classified by the SVM system based on learning from the training set derived from D-Histo analysis. A plot of the voxels 6650 from the SVM classification was checked against gold standard histology (H & E) images 6660, 6662, 6664 and 6666 of different areas (a, b, c and d) to confirm the correct classification. In this example, the voxels are primarily classified as tumor infiltration and tumor necrosis.

The above D-Histo based techniques may be used to monitor therapies such as immunotherapy. For example, the SVM classification described above or the D-Histo technique may be used to determine whether therapy for brain cancer is effective. The D-Histo technique may be applied to an MRI image before the therapy and then a second MRI image may be taken after the therapy. The D-histo technique allows more accurate classification of necrotic tumors that may demonstrate the effectiveness of the therapy. Such a process may be used to monitor other therapies such as for prostate cancer that may show the recession of prostate cancer cells after the application of therapy.

In an aspect, the D-Histo technique not only may assess nerve pathologies as explained above in relation to the DBSI method, but also can simultaneously assess the functioning of nerves, both in central and peripheral nerve systems. Longitudinal D-Histo was performed on experimental autoimmune encephalomyelitis (EAE) mouse optic nerves at a baseline (naive, before immunization), before, during, and after the onset of optic neuritis. In this cohort of mice, optic neuritis did not occur simultaneously for both eyes. Time 1 was defined as the day in which the first eye was affected and Time 2 as the day in which the second eye was impaired. Thus, Eye 1 is the eye affected at Time 1 and Eye 2 is the eye affected at Time 2. The D-Histo technique detected, differentiated, and quantified co-existing optic nerve pathologies in EAE mice.

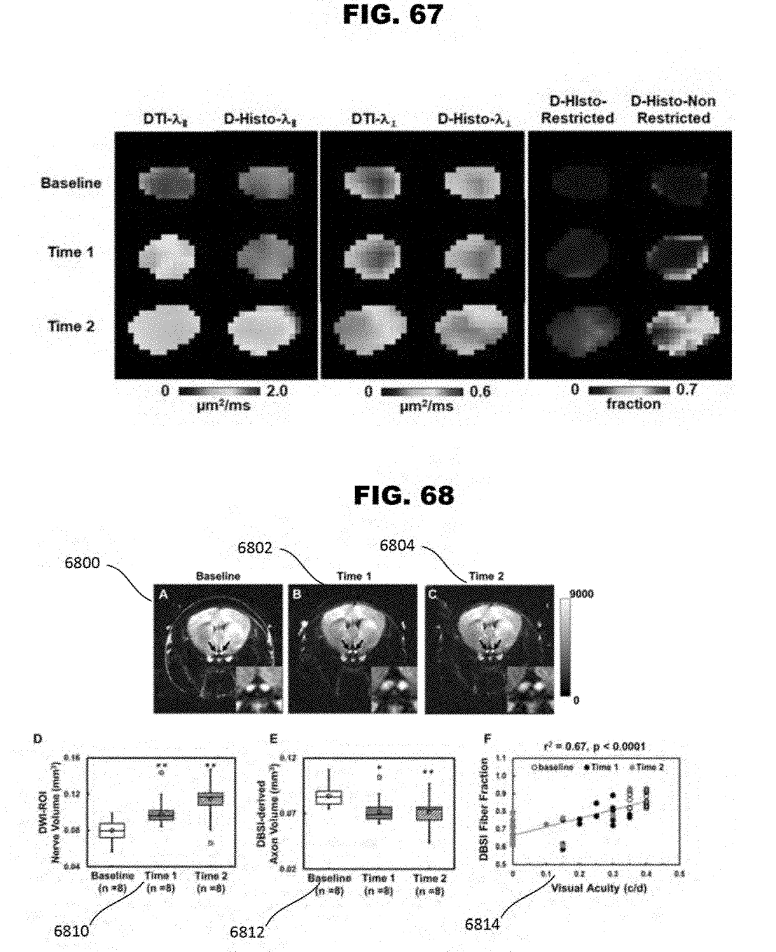

FIG. 67 shows DTI and D-Histo derived parametric maps of one representative EAE optic nerve (Eye 1) from baseline, Time 1, and Time 2. Decreased axial diffusivity (λ_{I}) and increased radial diffusivity (λ_{⊥}) in both DTI and D-Histo measurements suggest axonal injury and demyelination at Time 2. Using the D-Histo method distinguished and further quantified the extent of inflammatory cell infiltration (restricted diffusion fraction) and vasogenic edema (non-restricted diffusion fraction).

FIG. 68 shows diffusion-weighted images (DWI) acquired using the diffusion gradient applied perpendicular to the optic nerves (black arrows), at baseline (image 6800 taken before EAE induction), Time 1 (image 6802 taken at the onset of ON in the first eye), and Time 2 (image 6804 taken at the onset of ON in the second eye) from an representative EAE mouse. Optic nerve swelling was seen at Time 1 and 2 caused by inflammation associated increase in cellularity and edema. Significantly increased optic nerve volume was seen after optic neuritis (chart 6810, p<0.005). The corresponding D-Histo-derived axon volume (optic nerve volume × D-Histo anisotropic diffusion tensor fraction) suggested a significant axonal loss in optic nerves (chart 6812, p<0.05 and 0.005 for Time 1 and 2, respectively). D-Histo fiber fraction, reflecting effects of axonal loss and dilution effect of axonal density from inflammation, correlated well with visual acuity (chart 6814). In the charts 6810, 6812 and 6814, * indicates p<0.05 and ** indicates p<0.005.

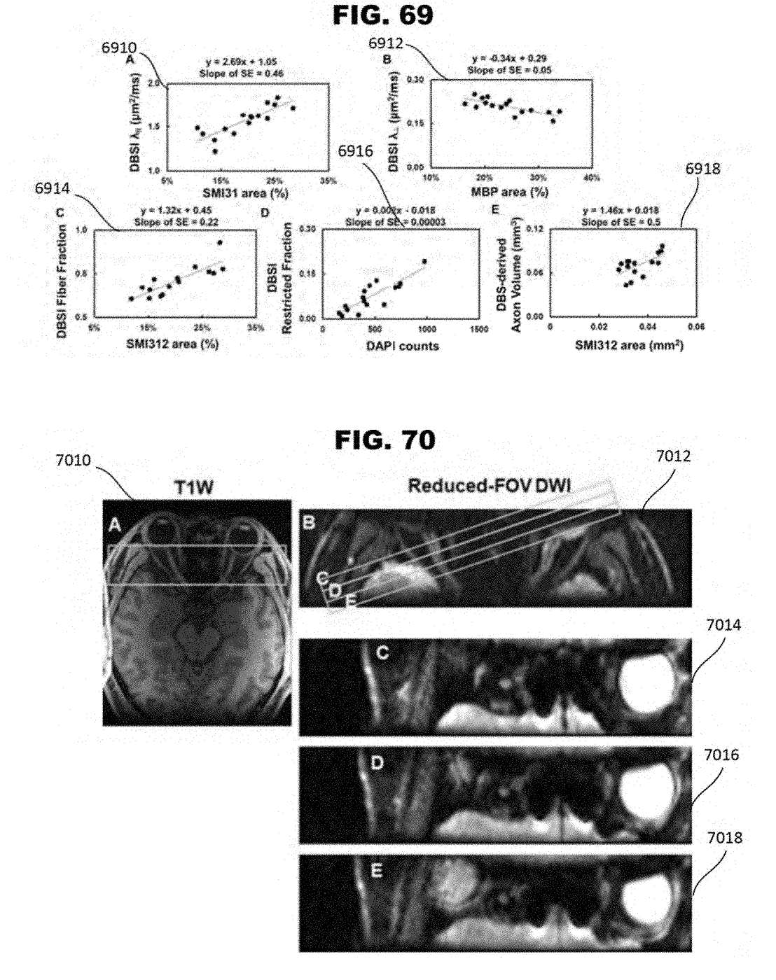

D-Histo derived pathological metrics were quantitatively validated by immunohistochemistry as demonstrated in the graphs shown in FIG. 69. A graph 6910 shows regression of SMI-31, MBP, SMI-312, DAPI counts and D-Histo derived λ_{|}. A graph 6912 shows regression of SMI-31, MBP, SMI-312, DAPI counts and D-Histo derived λ_{⊥}. A graph 6914 shows regression of SMI-31, MBP, SMI-312, DAPI counts and D-Histo derived fiber fraction. A graph 6916 shows regression of SMI-31, MBP, SMI-312, DAPI counts and restricted fraction. These graphs suggested D-Histo measurements were able to reflect specific pathologies in the optic nerves of EAE mice, respectively. The regression of D-Histo-derived axon volume correlated with SMI-312 area (in mm²). In contrast to SMI-312 area estimated as ratio of positive area over the total nerve cross-sectional area (% in graph 6914), a graph 6918 of the SMI-312 area in mm² reflects the extent of total axons without the dilution effect of inflammation. The SE is standard error in the graphs 6910, 6912, 6914, 6916, and 6918.

Diffusion-weighted MRI has been employed to assess optic nerve function in both control and EAE mice. The application of this technique in human central or peripheral nerves has never been reported. The tortuosity and different axonal packing between human and rodent nerves may contribute to the lack of successful human optic nerve diffusion MRI. Given the unique capability of D-Histo to assess axonal morphology and the pathological complexity of its surrounding, D-Histo can be applied to human central and peripheral nerves to accurately assess nerve function. Conventional diffusion-weighted MRI or diffusion tensor imaging may not be able to assess nerve function due to the morphology and axonal packing patterns.

FIG. 70 shows images that show the image planning to obtain the cross-sectional view of one optic nerve. A normal anatomic brain image 7010 was used to prescribe cross-sectional view of optic nerve using a reduced-field-of-view sequence as shown in an image 7012. The resulting diffusion-weighted MRI optic nerve segments in images 7014, 7016 and 7018 exhibit less distortion than conventional sequence used in FIG. 70A. In a healthy control subject, D-Histo and DTI techniques were performed on the optic nerve with and without visual stimulation in the form of an 8 Hz flashing checkerboard.

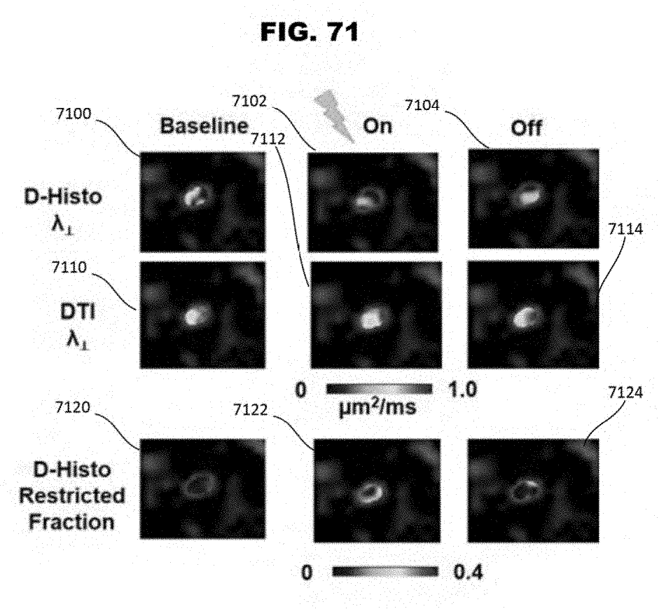

FIG. 71 shows a series of D-Histo maps 7100, 7102 and 7104 at a base line, during visual stimulation and without visual stimulation. FIG. 71 also shows a series of DTI maps 7110, 7102 and 7114 at a base line, during visual stimulation and without visual stimulation. FIG. 71 also shows a series of D-Histo restricted fraction maps 7120, 7122 and 7124 at a base line, during visual stimulation and without visual stimulation. These maps show that D-Histo derived radial diffusivity of optic nerve decreased upon visual stimulation, from 0.54 to 0.11 μm²/ms, while DTI derived radial diffusivity did not change remaining at 0.65 μm²/ms with and without visual stimulation. A D-Histo derived restricted isotropic diffusion tensor fraction increased from 0 at the baseline to 15% during visual stimulation. DTI does not compute restricted isotropic diffusion fraction.

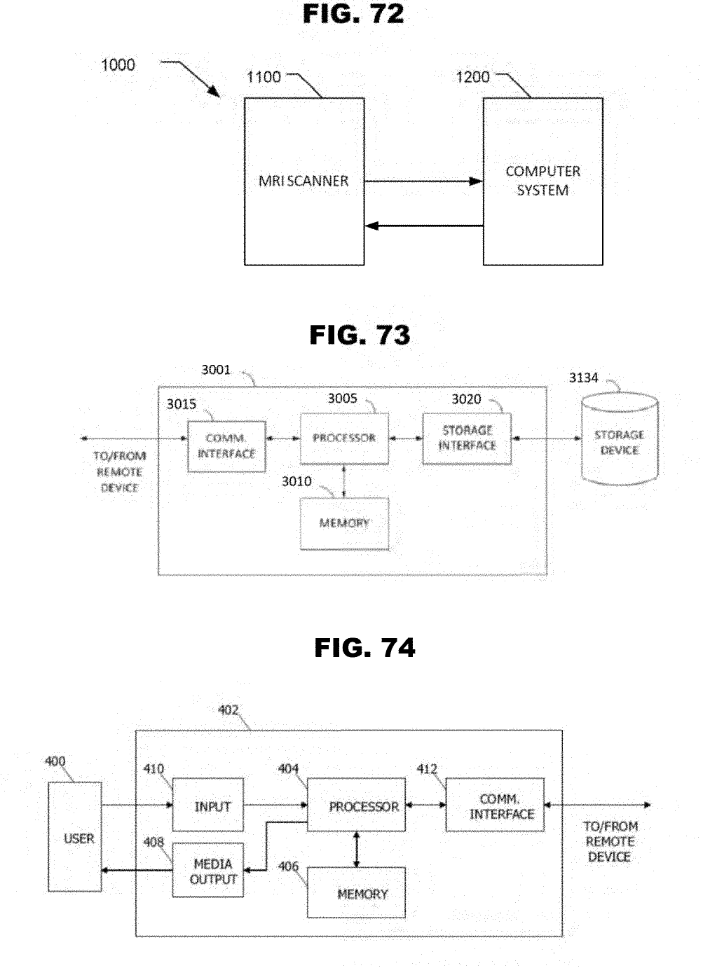

In various aspects, the methods described herein may be implemented using an MRI system. FIG. 72 is an illustration of an MRI imaging system 1000 in one aspect. As illustrated in FIG. 72, the MRI system 1000 may include an MRI scanner 1100 operatively coupled and/or in communication with a computer system 1200. In this aspect, the computer system 1200 is configured to receive data including, but not limited to, diffusion data, from the MRI scanner 1100, and is further configured to execute a plurality of stored executable instructions encoding one or more aspects of the D-Histo method as described herein above. In another aspect, the computer system 1200 may be further configured to operate the MRI scanner 1100 to obtain, for example, diffusion data by executing an additional plurality of stored executable instructions. The computer system 1200 may also be programmed to execute a machine learning application that learns classification of the MRI based images produced by the MRI scanner 1100 based on the D-Histo method. The computer system 1200 thus may produce histology maps based on the MRI images.

Although the present invention is described in connection with an exemplary imaging system environment, embodiments of the invention are operational with numerous other general purpose or special purpose imaging system environments or configurations. The imaging system environment is not intended to suggest any limitation as to the scope of use or functionality of any aspect of the invention. Moreover, the imaging system environment should not be interpreted as having any dependency or requirement relating to any one or combination of components illustrated in the exemplary operating environment. Examples of well-known imaging systems, environments, and/or configurations that may be suitable for use with aspects of the invention include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like.

Computer systems, as described herein, refer to any known computing device and computer system. As described herein, all such computer systems include a processor and a memory. However, any processor in a computer system referred to herein may also refer to one or more processors wherein the processor may be in one computing device or a plurality of computing devices acting in parallel. Additionally, any memory in a computer device referred to herein may also refer to one or more memories wherein the memories may be in one computing device or a plurality of computing devices acting in parallel.

The term processor, as used herein, refers to central processing units, microprocessors, microcontrollers, reduced instruction set circuits (RISC), application specific integrated circuits (ASIC), logic circuits, and any other circuit or processor capable of executing the functions described herein. The above are examples only, and are thus not intended to limit in any way the definition and/or meaning of the term “processor.”

As used herein, the term “database” may refer to either a body of data, a relational database management system (RDBMS), or to both. As used herein, a database may include any collection of data including hierarchical databases, relational databases, flat file databases, object-relational databases, object-oriented databases, and any other structured collection of records or data that is stored in a computer system. The above examples are example only, and thus are not intended to limit in any way the definition and/or meaning of the term database. Examples of RDBMSs include, but are not limited to including, Oracle® Database, MySQL, IBM® DB2, Microsoft® SQL Server, Sybase®, and PostgreSQL. However, any database may be used that enables the systems and methods described herein. (Oracle is a registered trademark of Oracle Corporation, Redwood Shores, Calif.; IBM is a registered trademark of International Business Machines Corporation, Armonk, N.Y.; Microsoft is a registered trademark of Microsoft Corporation, Redmond, Washington; and Sybase is a registered trademark of Sybase, Dublin, Calif.)

In one embodiment, a computer program is provided to enable the data processing of the D-Histo method as described herein above, and this program is embodied on a computer readable medium. In an example embodiment, the computer system is executed on a single computer system, without requiring a connection to a server computer. In a further embodiment, the computer system is run in a Windows® environment (Windows is a registered trademark of Microsoft Corporation, Redmond, Wash.). In yet another embodiment, the computer system is run on a mainframe environment and a UNIX® server environment (UNIX is a registered trademark of X/Open Company Limited located in Reading, Berkshire, United Kingdom). Alternatively, the computer system is run in any suitable operating system environment. The computer program is flexible and designed to run in different environments without compromising any major functionality. In some embodiments, the computer system includes multiple components distributed among a plurality of computing devices. One or more components may be in the form of computer-executable instructions embodied in a computer-readable medium.

The computer systems and processes are not limited to the specific embodiments described herein. In addition, components of each computer system and each process can be practiced independent and separate from other components and processes described herein. Each component and process also can be used in combination with other assembly packages and processes.

In one embodiment, the computer system may be configured as a server system. FIG. 73 illustrates an example configuration of a server system 3001 used to receive measurements from the MRI scanner 1100 (not illustrated). Referring again to FIG. 73, server system 3001 may also include, but is not limited to, a database server. In this example embodiment, server system 3001 performs all of the steps used to implement the MRI imaging method as described herein above.

In this aspect, the server system 3001 includes a processor 3005 for executing instructions. Instructions may be stored in a memory area 3010, for example. The processor 3005 may include one or more processing units (e.g., in a multi-core configuration) for executing instructions. The instructions may be executed within a variety of different operating systems on the server system 3001, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or any other suitable programming languages).

The processor 3005 is operatively coupled to a communication interface 3015 such that server system 3001 is capable of communicating with a remote device, such as the MRI scanner 1100, a user system, or another server system 301. For example, communication interface 3015 may receive requests (e.g., requests to provide an interactive user interface to receive sensor inputs and to control one or more devices of system 1000 from a client system via the Internet.

Processor 3005 may also be operatively coupled to a storage device 3134. Storage device 3134 is any computer-operated hardware suitable for storing and/or retrieving data. In some embodiments, storage device 3134 is integrated in server system 3001. For example, server system 3001 may include one or more hard disk drives as storage device 3134. In other embodiments, storage device 3134 is external to server system 3001 and may be accessed by a plurality of server systems 3001. For example, storage device 3134 may include multiple storage units such as hard disks or solid-state disks in a redundant array of inexpensive disks (RAID) configuration. Storage device 3134 may include a storage area network (SAN) and/or a network attached storage (NAS) system.

In some embodiments, processor 3005 is operatively coupled to storage device 3134 via a storage interface 3020. Storage interface 3020 is any component capable of providing processor 3005 with access to storage device 3134. Storage interface 3020 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 3005 with access to storage device 3134.

Memory area 3010 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), non-volatile RAM (NVRAM), registers, hard disk memory, a removable disk, a CD-ROM, or any other form of computer-readable storage medium known in the art. The above memory types are exemplary only, and are thus not limiting as to the types of memory usable for storage of a computer program.

In another embodiment, the computer system may be provided in the form of a computing device, such as a computing device 402 (shown in FIG. 74). Computing device 402 includes a processor 404 for executing instructions. In some embodiments, executable instructions are stored in a memory area 406. Processor 404 may include one or more processing units (e.g., in a multi-core configuration). Memory area 406 is any device allowing information such as executable instructions and/or other data to be stored and retrieved. Memory area 406 may include one or more computer-readable media.

In another embodiment, the memory included in the computing device 402 may include a plurality of modules. Each module may include instructions configured to execute using at least one processor. The instructions contained in the plurality of modules may implement at least part of the method for simultaneously regulating a plurality of process parameters as described herein when executed by the one or more processors of the computing device. Non-limiting examples of modules stored in the memory of the computing device include: a first module to receive measurements from one or more sensors and a second module to control one or more devices of the MRI imaging system 1000.

Computing device 402 also includes one media output component 408 for presenting information to a user 400. Media output component 408 is any component capable of conveying information to user 400. In some embodiments, media output component 408 includes an output adapter such as a video adapter and/or an audio adapter. An output adapter is operatively coupled to processor 404 and is further configured to be operatively coupled to an output device such as a display device (e.g., a liquid crystal display (LCD), organic light emitting diode (OLED) display, cathode ray tube (CRT), or “electronic ink” display) or an audio output device (e.g., a speaker or headphones).

In some embodiments, client computing device 402 includes an input device 410 for receiving input from user 400. Input device 410 may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component 408 and input device 410.

Computing device 402 may also include a communication interface 412, which is configured to communicatively couple to a remote device such as server system 302 or a web server. Communication interface 412 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory 406 are, for example, computer-readable instructions for providing a user interface to user 400 via media output component 408 and, optionally, receiving and processing input from input device 410. A user interface may include, among other possibilities, a web browser and an application. Web browsers enable users 400 to display and interact with media and other information typically embedded on a web page or a website from a web server. An application allows users 400 to interact with a server application.

Exemplary embodiments of methods, systems, and apparatus for use in diffusion basis spectrum imaging/D-Histo are described above in detail. The methods, systems, and apparatus are not limited to the specific embodiments described herein but, rather, operations of the methods and/or components of the systems and/or apparatus may be utilized independently and separately from other operations and/or components described herein. Further, the described operations and/or components may also be defined in, or used in combination with, other systems, methods, and/or apparatus, and are not limited to practice with only the systems, methods, and apparatus described herein.

The order of execution or performance of the operations in the embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

It will be understood by those of skill in the art that information and signals may be represented using any of a variety of different technologies and techniques. For example, data, instructions, commands, information, signals, bits, symbols, and/or chips may be represented by voltages, currents, electromagnetic waves, magnetic fields or particles, optical fields or particles, or any combination thereof. Similarly, the various illustrative logical blocks, modules, circuits, and algorithm operations described herein may be implemented as electronic hardware, computer software, or a combination of both, depending on the application and the functionality. Moreover, the various logical blocks, modules, and circuits described herein may be implemented or performed with a general purpose computer, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. Exemplary general-purpose processors include, but are not limited to only including, microprocessors, conventional processors, controllers, microcontrollers, state machines, or a combination of computing devices.

When introducing elements of aspects of the invention or embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of classifying microstructures in a tissue volume, the method comprising:
taking an MRI image of the tissue volume by an MRI scanner;
determining diffusion tensor components of water molecules within a voxel derived from the MRI image via a processor coupled to the MRI scanner;
determining apparent diffusion coefficients of the water molecules with diffusion tensor components falling in a predetermined range associated with a microstructure via the processor; and
identifying the microstructure in the voxel derived from the MRI image based on classified diffusion tensor components where the apparent diffusion coefficients fall in the predetermined range via the processor.

2. The method of claim 1, wherein the microstructure is a cancerous microstructure.

3. The method of claim 1, wherein the microstructure is a non-cancerous microstructure.

4. The method of claim 1, further comprising generating an image map of the tissue volume from the voxel showing a presence of the microstructure on an electronic display.

5. The method of claim 1, wherein determining the apparent diffusion coefficients and identifying the microstructure are performed at a first time, and the method further comprises:
treating the tissue volume with a treatment method;
determining the apparent diffusion coefficients of the water molecules with diffusion tensor components falling in a predetermined range associated with the microstructure at a second time; and
identifying the microstructure in the voxel derived from the MRI image based on classified diffusion tensor components where the apparent diffusion coefficients fall in the predetermined range at the second time; and
comparing the identified microstructure at the first time with identified microstructure at the second time to determine an effectiveness of the treatment method.

6. The method of claim 1, further comprising:
creating a set of training data from the identified microstructure and the MRI image; and
training a machine learning system to identify the microstructure from an MM image based on the set of training data.

7. The method of claim 1, further comprising placing a biopsy needle in the tissue volume at the voxel including the identified microstructure.

8-24. (canceled)

25. The method of claim 1, wherein the tissue volume is taken from a prostate.

26. The method of claim 3, wherein the non-cancerous microstructure includes at least one of stroma, inflammation, lumen or normal prostate tissue.

27. The method of claim 26, wherein the classified diffusion tensor components are anisotropic diffusion for stroma, wherein the classified diffusion tensor components are a highly restricted isotropic diffusion and a predetermined range for inflammation is between 0 and 0.1, wherein the classified diffusion tensor components are a non-restricted isotropic diffusion and a predetermined range for lumen or normal prostate tissue is between 0.7 and 3.5.

28. The method of claim 26, wherein the classified diffusion tensor components are a restricted isotropic diffusion a predetermined range for prostate cancer is between 0.1 to 0.7.

29. The method of claim 2, wherein the tissue volume is taken from a brain.

30. The method of claim 29, wherein the cancerous microstructure is one of infiltrating tumor cells, necrotic tumor cells, immune cells, and dense (viable) tumors.

31. The method of claim 30, wherein the classified diffusion tensor components are a highly restricted fraction and a predetermined range is between 0 to 0.2 for low-grade glioma and immune cells, wherein the classified diffusion tensor components are a restricted fraction and a predetermined range for dense (viable) tumors is between 0.2 to 1, and the classified diffusion tensor components are a hindered fraction tensor and a predetermined range is between 1.0 to 1.5 for necrotic tumor cells.

32. The method of claim 3, wherein the non-cancerous microstructure is white matter and the classified diffusion tensor components are a high fiber fraction.

33. The method of claim 1, wherein the tissue volume is taken from one of a cervix, a breast, cardiac tissue, a pancreas, a bladder, a kidney, and a nerve.
